(19) United States
(12) Patent Application Publication
(10) Pub. No.: US 2015/0150782 A1
(43) Pub. Date: Jun. 4, 2015

(54) HAIR CARE COMPOSITIONS INCLUDING OSAGE ORANGE

(71) Applicant: Todd Johnson, Burlington, IA (US)

(72) Inventor: Todd Johnson, Burlington, IA (US)

(21) Appl. No.: 14/096,829

(22) Filed: Dec. 4, 2013

(51) Int. Cl. A61K 8/97; A61Q 5/06; A61Q 5/12; A61K 8/19; A61K 8/41

(52) U.S. Cl. CPC A61K 8/97; A61K 8/19; A61K 8/41; A61Q 5/12; A61Q 5/06

(57) ABSTRACT

The present invention relates to hair care products including Osage Orange in the form of the botanical extract, seed oil, or combinations of the same. The compositions of the invention synergistically react to stabilize the compositions and provide anti-oxidant, moisturizing, and conditioning benefits to hair and the scalp, while cleansing the hair and not removing essential oils. In further embodiments, the compositions of the present invention can be used for healing hair or styling hair, while providing the benefit of moisturizing and providing anti-oxidants. Moreover, embodiments of the invention can be substantially free of harsh chemical preservatives and chemically synthesized anti-oxidants.

### FIELD OF THE INVENTION

The present invention relates to the use of Osage Orange in hair care products. In particular, the present invention relates to hair care products containing Osage Orange botanical extract and/or Osage Orange seed oil. More particularly the present invention relates to shampoos and cleansing conditioners containing Osage Orange botanical extract and/or Osage Orange seed oil.

### BACKGROUND OF THE INVENTION

Today it is common practice for people to wash their hair with a shampoo and then to apply a conditioner or conditioning treatment. Typical shampoos are a mixture of chemicals intended to remove dirt, oil, and sweat from the hair and scalp. Traditionally, many chemicals used in shampoos are necessarily harsh, such as sodium lauryl sulfate, and remove the soils, dirt, and other oils intended for removal, in addition to the hair's essential oils. This results in hair that is dry, dull, lusterless, rough, frizzy, and lacking body or other desired characteristics. Moreover, many existing hair treatments, such as coloring, perming, straightening, and curling, damage the hair. This can result in the hair becoming brittle, dull, and having split ends. When the hair has structural damage it can be more difficult comb and style. Conditioners and conditioning treatments are intended to replace the essential oils washed out of the hair by shampoo so that the hair's health, shine, and body can be restored.

Clearly, it is counterintuitive to remove the essential oils by harsh cleansing and then attempt to replace the same essential oils. Rather, it would be ideal to cleanse the hair with a hair care product that does not remove oils in the first place. Development of a hair cleanser that can clean the hair without removing essential oils would greatly improve the field of hair treatments and satisfy a long felt need of the public.

Another problem that some people are faced with is folliculitis. Folliculitis is the medical term for the infection and inflammation of hair follicles. The symptoms of this condition differ according to the type of infection. The indications of folliculitis in general can be of two variants, superficial folliculitis and deep folliculitis. Superficial folliculitis occurs in the upper part of the hair follicle, and the indications are: reddened or inflamed skin, sore or small red itchy bumps around the hair follicle, and blisters that burst and crust over. Deep folliculitis occurs in the deeper portion inside the skin, and the entire hair follicle is affected. A few of its symptoms include inflammation and scarring of the affected area. For mild cases of each folliculitis, the symptoms usually can be managed with regular cleaning of the scalp and hair, using a mild shampoo that contains ingredients with either antifungal, antimicrobial, or anti-inflammatory properties.

Current attempts to remedy the aforementioned problems have included shampoos that include conditioner, shampoos with anti-inflammatory agents such as salicylic acid, and anti-dandruff shampoos with antifungal agents like ketoconazole or ciclopirox. Additionally, shampoos with antimicrobial ingredients such as selenium sulfide can be useful for treating folliculitis. Many of the shampoos and conditioners intended for treating folliculitis contain synthesized anti-oxidants. However, the use of chemically synthesized anti-oxidants such as butylated hydroxyl anisole (BHA), butylated hydroxyl toluene (BHT), and tert-butyl hydroquinone (BHQ) has fallen out of favor for health and environmental reasons. As such, it is desirable to use natural product anti-oxidants in hair care products. Moreover, there is a need in the hair care market for products that avoid the use of harsh chemical preservatives such as chloroisothiazolinone, methylchloroisothiazolinone, methylisothiazolinone, and tricolsan. In fact, regulatory bodies and watchdog groups have recently questioned or have forbidden the use of some of these chemical preservatives.

Other attempts to remedy the aforementioned problems have been to incorporate vegetable, nut, and/or plant oils as emollients and to moisturize the hair. Such attempts have been made because these ingredients contain unsaturated fatty acids and lipids—in particular Omega-6 fatty acid (linoleic acid). However, it is also well known that the desired unsaturated fatty acids and lipids are very susceptible to oxidation leading to the formation of peroxide and hydroperoxides. Peroxides and hydroperoxides are not only unstable but damage hair. Thus, attempts to remedy the aforementioned problems have been less than optimal, and in some instances, have further exacerbated the problems associated with hair damage.

Accordingly, a primary objective of the present invention is the provision of an improved hair care product that both cleans and conditions hair.

Another objective of the present invention to develop hair care products with anti-oxidants that inhibit the oxidation of unsaturated fatty acids and lipids, thereby enhancing the performance and stability of hair care products and protecting the hair from free radical damage due to the decomposition of peroxide and hydroperoxide.

Another objective of the invention is to provide hair care products that do not remove the essential oils from hair and that does not damage the hair.

Still another objective of the invention is to provide a conditioner that can be used for cleansing the hair in addition to treating damaged hair.

A further objective of the invention is to provide hair care products that are free of the harsh chemicals, including, chloroisothiazolinone, methylchloroisothiazolinone, methylisothiazolinone, sodium lauryl sulfate, tricolsan, and formaldehyde donors such as DMDM hydantoin when paired with iodopropynyl butylcarbamate and paraben.

A still further objective of the invention is to provide hair care products that are free of chemically synthesized anti-oxidants including, but not limited to, butylated hydroxyl anisole (BHA), butylated hydroxyl toluene (BHT), and tert-butyl hydroquinone (BHQ).

Yet another objective of the invention is to provide a hair care product that overcomes the problems in the art.

Another objective of the present invention is the provision of a hair care product that is economical to manufacture and safe to use.

Other objects, advantages and features of the present invention will become apparent from the following specification.

### SUMMARY OF THE INVENTION

An advantage of the invention is the ability to cleanse hair without removing essential oils and without damaging the hair. It is an advantage of the present invention that the Osage Orange provides antioxidants, inhibits the oxidation of unsaturated fatty acids and lipids, thereby protecting the hair and scalp from free radical damage. Moreover, the Osage Orange acts synergistically with the other ingredients to stabilize the composition.

Accordingly, an embodiment of the present invention is found in hair care products comprised of Osage Orange, an antistatic agent comprising a cationic quaternary ammonium, a conditioning agent comprising a fatty amine, a surfactant, and an aqueous carrier. In another aspect of the invention, the ratio of conditioning agent to antistatic agent is between about 3:1 and about 1:3. In a further aspect of the invention the ratio of water to the combination of conditioning agent and antistatic agent is between about 400:24 and about 750:24. In still another aspect of the invention, the ratio of total plant oil to the combination of antistatic agent and conditioning agent is between about 1:1 and about 4:5. In another embodiment of the invention, the hair care composition may comprise Osage Orange, a fixative, a hydrotrope, an emulsifier, a thickener, and an aqueous carrier. In still a further embodiment of the invention, the hair care composition may comprise Osage Orange, a silicone compound, and at least one functional ingredient. In an aspect of the invention, the hair care product may be a shampoo, cleansing conditioner, a styling gel, or a healing treatment.

While multiple embodiments are disclosed, still other embodiments of the present invention will become apparent to those skilled in the art from the following detailed description, which shows and describes illustrative embodiments of the invention. Accordingly, the detailed description is to be regarded as illustrative in nature and not restrictive.

### DETAILED DESCRIPTION OF THE INVENTION

The present invention more specifically relates to compositions for improving hair condition, hair appearance, and hair care product performance, and more particularly to compositions including prenylated isoflavonoid compounds, Osage Orange extract, and Osage Orange seed oil, that are effective for imparting direct benefit to the scalp and hair, protection of the hair and scalp from free radical damage, and stabilization of ingredients commonly used in the hair care industry—especially Omega-3, Omega-6, and Omega-9 fatty acids—and use of a novel source of an oil as an emollient containing Vitamin E and other beneficial phytonutrients. The embodiments of this invention are not limited to particular hair care products, methods of cleaning and or treating hair, or methods of preparing hair care products, which can vary and are understood by skilled artisans. It is further understood that all terminology used herein is for the purpose of describing particular embodiments only, and is not intended to be limiting in any manner or scope.

### DEFINITIONS

So that the present invention may be more readily understood, certain terms are first defined. Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments of the present invention without undue experimentation. The preferred materials and methods are described herein. In describing and claiming the embodiments of the present invention, the following terminology will be used in accordance with the definitions set out below.

For example, as used in this specification and the appended claims, the singular forms “a,” “an” and “the” can include plural referents unless the content clearly indicates otherwise. Further, all units, prefixes, and symbols may be denoted in its SI accepted form. Numeric ranges recited within the specification are inclusive of the numbers defining the range and include each integer within the defined range.

The recitation of numerical ranges by endpoints includes all numbers subsumed within that range (e.g., 1 to 5 includes 1, 1.3, 2, 2.72, 3, 3.60, 4, and 5).

The term “about,” as used herein, refers to variation in the numerical quantity that can occur, for example, through typical measuring and liquid handling procedures used for making concentrates or use solutions in the real world; through inadvertent error in these procedures; through differences in the manufacture, source, or purity of the ingredients used to make the compositions or carry out the methods; and the like. The term “about” also encompasses amounts that differ due to different equilibrium conditions for a composition resulting from a particular initial mixture. Whether or not modified by the term “about,” the claims include equivalents to the quantities. Moreover, whether or not values in the specification are modified by the term “about,” the term “about” should be understood to apply to those values when desired.

The term “actives” or “percent actives” or “percent by weight actives” or “actives concentration” are used interchangeably herein and refers to the concentration of those ingredients involved in cleaning expressed as a percentage minus inert ingredients such as water or salts. It is also sometimes indicated by a percentage in parentheses, for example, “chemical (10%).”

As used herein, the term “alkyl” or “alkyl groups” refers to saturated hydrocarbons having one or more carbon atoms, including straight-chain alkyl groups (e.g., methyl, ethyl, propyl, butyl, pentyl, hexyl, heptyl, octyl, nonyl, decyl, etc.), cyclic alkyl groups (or “cycloalkyl” or “alicyclic” or “carbocyclic” groups) (e.g., cyclopropyl, cyclopentyl, cyclohexyl, cycloheptyl, cyclooctyl, etc.), branched-chain alkyl groups (e.g., isopropyl, tert-butyl, sec-butyl, isobutyl, etc.), and alkyl-substituted alkyl groups (e.g., alkyl-substituted cycloalkyl groups and cycloalkyl-substituted alkyl groups).

Unless otherwise specified, the term “alkyl” includes both “unsubstituted alkyls” and “substituted alkyls.” As used herein, the term “substituted alkyls” refers to alkyl groups having substituents replacing one or more hydrogens on one or more carbons of the hydrocarbon backbone. Such substituents may include, for example, alkenyl, alkynyl, halogeno, hydroxyl, alkylcarbonyloxy, arylcarbonyloxy, alkoxycarbonyloxy, aryloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), imino, sulfhydryl, alkylthio, arylthio, thiocarboxylate, sulfates, alkylsulfinyl, sulfonates, sulfamoyl, sulfonamido, nitro, trifluoromethyl, cyano, azido, heterocyclic, alkylaryl, or aromatic (including heteroaromatic) groups.

In some embodiments, substituted alkyls can include a heterocyclic group. As used herein, the term “heterocyclic group” includes closed ring structures analogous to carbocyclic groups in which one or more of the carbon atoms in the ring is an element other than carbon, for example, nitrogen, sulfur or oxygen. Heterocyclic groups may be saturated or unsaturated. Exemplary heterocyclic groups include, but are not limited to, aziridine, ethylene oxide (epoxides, oxiranes), thiirane (episulfides), dioxirane, azetidine, oxetane, thietane, dioxetane, dithietane, dithiete, azolidine, pyrrolidine, pyrroline, oxolane, dihydrofuran, and furan.

As used herein, the phrase “ambient conditions” means conditions typical of normal room temperature and pressure including fluctuations, with a pressure of about 1 atmosphere and a temperature between about 40° F. and about 95° F.

The term “microemulsion” as used herein, refers to a thermodynamically stable liquid dispersion of one liquid phase into another that is stabilized by an interfacial film of surfactant. According to the invention, the microemulsion forms an interface with the surfactant between the two immiscible phases of oil and water.

As used herein, the phrase “plant” or “plant product” includes any plant substance or plant-derived substance. Plant products include, but are not limited to, seeds, nuts, nut meats, cut flowers, plants or crops grown or stored in a greenhouse, house plants, and the like.

As used herein, the term “soil” refers to a non-polar oily substance which may or may not contain particulate matter such as mineral clays, sand, natural mineral matter, carbon black, graphite, kaolin, environmental dust, etc.

As used herein, the term “substantially free” refers to compositions completely lacking the component or having such a small amount of the component that the component does not affect the performance of the composition. The component may be present as an impurity or as a contaminant and shall be less than 0.5 wt-%. In another embodiment, the amount of the component is less than 0.1 wt-% and in yet another embodiment, the amount of component is less than 0.01 wt-%.

As used herein, the term “suitable for application to human hair” means that the compositions or components thereof are acceptable for use in contact with human hair, the scalp, and skin without undue toxicity, incompatibility, instability, allergic response, and the like.

As used herein, the phrase “total plant oil” refers to the total amount of plant-derived oils and extracts, including extracts and oils from Osage Orange and excluding plant-derived juices and butters.

As used herein, the phrase “water soluble” means that the material is soluble in water in the present composition. In general, the material should be soluble at 25° C. at a concentration of about 0.1 wt. % of the water, alternatively at about 1 wt. %, alternatively at about 5 wt. %, and alternatively at about 15 wt. %.

The term “weight percent,” “wt. %,” “wt-%,” “percent by weight,” “% by weight,” and variations thereof, as used herein, refer to the concentration of a substance as the weight of that substance divided by the total weight of the composition and multiplied by 100. It is understood that, as used here, “percent,” “%,” and the like are intended to be synonymous with “weight percent,” “wt-%,” etc.

The compositions and methods of the present invention may comprise, consist essentially of, or consist of the components and ingredients of the present invention as well as other ingredients described herein. As used herein, “consisting essentially of” means that the methods, systems, apparatuses and compositions may include additional steps, components or ingredients, but only if the additional steps, components or ingredients do not materially alter the basic and novel characteristics of the claimed methods, systems, apparatuses, and compositions.

### Hair Care Compositions

Surprisingly it was found that hair care products comprised of Osage Orange extract and/or oil have greater stability and inhibit the oxidation of saturated fatty acids and lipids. Thus, embodiments of the present invention employ chemicals capable of cleansing hair with desired moisturizing and antioxidant properties while inhibiting the oxidation of unsaturated fatty acids and lipids thereby protecting the hair from free radical damage caused by peroxide and hydroperoxide moieties. Such compositions are particularly effective at cleansing, moisturizing, and conditioning hair, while not damaging hair as existing cleansing conditioners and shampoos do. Moreover, the compositions of the present invention can be incorporated in a variety of other hair care products including, for example, styling gels and hair sprays.

Osage Orange

The compositions of the invention comprise prenylated isoflavonoid compounds isolated from Osage Orange (Maclura Pomifera) and the botanical extract of Osage Orange. The prenylated isoflavonoid compounds isolated from Osage Orange can be in the form of an extract or from Osage Orange seed oil. The prenylated isoflavonoid compounds isolated from the Osage Orange and the botanical extract of the Osage Orange, have been used in skin care products. This use is described by Gruber, U.S. Pat. App. Pub. No. 2010/0143451 and Orlow, U.S. Pat. App. Pub. No. 2012/0196926, each of which is incorporated herein in its entirety. Despite the use in skin care products, no one has recognized the benefits that the botanical extract of the Osage Orange and prenylated isoflavonoids compounds isolated from the Osage Orange can convey to hair care products. Furthermore, the isolated prenylated isoflavonoids and the extract of the Osage Orange can impart benefit not only to the follicles and hair itself, but they also interact synergistically to enhance performance of ingredients commonly found in hair care products. While not wishing to be bound by this theory, Applicant believes that this synergistic performance enhancement is in part due to the anti-oxidant, antimicrobial, and anti-fungal properties of the Osage Orange botanical extract and isolated prenylated isoflavonoids of Osage Orange.

Isolated prenylated isoflavonoids and/or the extract of the Osage Orange have significant anti-oxidant, anti-inflammatory, antifungal, antiviral, and antimicrobial properties. In an aspect of the invention, it can be used for treating or preventing folliculitis when formulated with hair care products like shampoo, conditioner, styling gel, healing treatment, and hair spray. Moreover, in another aspect of the invention, the anti-oxidants contained in the prenylated isoflavonoids compounds isolated from the Osage Orange and the botanical extract of the Osage Orange can protect the hair and scalp from free radical damage. As such, the present invention has application for treating and/or preventing free radical damage to the hair and scalp when formulated with hair care products like shampoo, conditioner, styling gel, and hair spray. Surprisingly Applicant has found that the antioxidant properties of the isolated prenylated isoflavonoids inhibit the oxidation of unsaturated fatty acids and lipids. This is in contrast to existing hair care products, whose antioxidants typically cause the oxidation of fatty acids and lipids. Thus, the present invention not only benefits the hair and scalp for aforementioned reasons but also inhibits the oxidation of fatty acids and lipids, which lead to hair damage caused by peroxide and hydroperoxide moieties. This also results in the stabilization of several ingredients commonly found in hair care products, as they remain un-oxidized. In this respect, isolated prenylated isoflavonoids and/or the extract of the Osage Orange acts as natural preservative in the hair care products of the present invention. This alleviates the need for harsh chemical preservatives, which are typically required to prevent microbial growth in hair care products. In particular, the present invention demonstrates that microbial growth limits are not exceeded when isolated prenylated isoflavonoids and/or the extract of the Osage Orange is formulated in hair care products including shampoo, conditioner, styling gel, healing treatment, and hair spray.

Osage Orange is included in compositions of the present invention, preferably from about 9 mg/kg to about 13,750 mg/kg, more preferably from about 10 mg/kg to about 12,650 mg/kg, and most preferably from about 11 mg/kg to about 11,770 mg/kg. In particular embodiments, the Osage Orange is included in the compositions preferably from about 9 mg/kg to about 15 mg/kg, more preferably from about 10.2 mg/kg to about 13.8 mg/kg, and most preferably from about 11.1 mg/kg to about 12.8 mg/kg. In particular embodiments, the Osage Orange is included in the compositions preferably from about 445 mg/kg to about 750 mg/kg, more preferably from about 500 mg/kg to about 690 mg/kg, and most preferably from about 555 mg/kg to about 640 mg/kg. In particular embodiments, the Osage Orange is included in the compositions preferably from about 8,250 mg/kg to about 13,750 mg/kg, more preferably from about 9,350 mg/kg to about 12,650 mg/kg, and most preferably from about 10,230 mg/kg to about 11,770 mg/kg. In particular embodiments, the Osage Orange is included in the compositions preferably from about 1,500 mg/kg to about 2,500 mg/kg, more preferably from about 1,700 mg/kg to about 2,300 mg/kg, and most preferably from about 1,860 mg/kg to about 2,140 mg/kg. It may be included as a botanical extract, seed oil, or the combination of both.

Additional Plant-Derived Ingredients

In addition to the use of Osage Orange oil and/or extract, the invention may optionally include additional plant-derived ingredients, such as oils, extracts, and juices. Typically, the extracts, juices, and oils are provided by extraction of the entire plant. However, in individual cases it may also be preferable to produce the extracts solely from the blossoms, seeds, and/or leaves of the plant.

Selection of the additional plant-derived oil is not limited in particular. Suitable plant-derived oils can include broadly any vegetable, fruit, and/or nut oils. Examples include, but are not limited to, acorn oil, allanblackia oil, almond oil, aloe vera oil, apricot kernel oil, arachis oil, arnica oil, argan oil, avocado oil, babassu oil, baobab oil, black seed oil, blackberry seed oil, blackcurrant seed oil, blueberry seed oil, borage oil, brazil nut oil, calendula oil, camelina oil, camellia kissi seed oil, camellia seed oil, castor oil, cherry kernel oil, Chinese gimlet oil, coconut oil, corn oil, cottonseed oil, evening primrose oil, flaxseed oil, germ oil, grapefruit oil, grapeseed oil, hazelnut oil, hempseed oil, Japanese nutmeg oil, Japan gimlet oil, jojoba oil, lemon seed oil, lime seed oil, linseed oil, kukui nut oil, macadamia nut oil, maize oil, meadowfoam oil, melon seed oil, moringa oil, Oleum Camelliae, olive oil, orange seed oil, palm oil, palm kernel oil, papaya seed oil, paraffin oil, passion seed oil, peach kernel oil, peanut oil, perilla oil, pine-needle oil, plum oil, pomegranate seed oil, poppy seed oil, pumpkin seed oil, rapeseed (or canola) oil, red raspberry seed oil, rice bran oil, rosehip oil, safflower oil, seabuckthorn oil, sesame oil, soybean oil, strawberry seed oil, sunflower oil, sweet almond oil, tea seed oil, walnut oil, and wheat germ oil. Particularly suitable are those oils that contain one or more of the following essential oils: omega-3, omega-6, omega-9, palmitic acid, stearic acid, and similar lipids. Preferably, an additional plant-derived oil will contain at least three essential oils. Particularly suitable additional plant-derived oils include, avocado oil, grapeseed oil, and jojoba oil.

Suitable extracts for the compositions of the invention may be extracts from acorn, almond, aloe vera, apricot, birch, burdock root, chamomile, coconut, coltsfoot, cucumber, ginseng, ginger root, grapefruit, green tea, hawthorn, henna, hops, horse chestnut, horsetail, juniper, kiwi, lady's smock, lime, lime blossom, mallow, mango, marsh mallow, melissa, melon, meristem, oak bark, orange, pomegranate, restharrow, rosemary, sage, sandalwood, stinging nettle, thyme, vanilla, wheat, wild thyme, witch hazel, and yarrow.

Suitable juices for the compositions of the invention may be juices from aloe vera, apples, coconut, grapefruit, lemon, lime, orange, and pomegranate.

In an embodiment of the invention, it may be desirable to include at least one additional plant-derived ingredient, in a further embodiment at least two additional plant-derived ingredients, and still a further embodiment at least three additional plant-derived ingredients. When included in compositions of the present invention, the additional plant-derived ingredients may be present, individually or in combination, preferably from about 0.1 wt. % to about 15 wt. %, more preferably 0.25 wt. % to about 12 wt. %, and most preferably 0.5 wt. % to about 10 wt. %.

Anti-Dandruff Agents

The compositions of the present invention may optionally contain anti-dandruff agents. Suitable anti-dandruff agents include, but are not limited to, pyridinethione salts, azoles, selenium sulfide, particulate sulfur, keratolytic agents, and mixtures thereof. When included in compositions of the present invention, the anti-dandruff agent selected should be physically and chemically compatible with the other components of the composition. It should not unduly impair product stability, aesthetics, or performance. Those of skill in the art will be able to select such an anti-dandruff agent. When present in the composition, the anti-dandruff agent is present preferably from about 0.01% to about 5%, more preferably from about 0.1% to about 3%, and most preferably from about 0.3% to about 2%, by weight of the composition.

Antistatic Agent

The compositions of the invention comprise one or more antistatic agents. Preferred antistatic agents for use in compositions of the present invention comprise cationic quaternary ammonium compounds. When used in compositions according to the invention the antistatic agent will be present preferably from about 0.2 wt. % to about 3 wt. %, more preferably from about 0.3 wt. % to about 2.7 wt. %, and most preferably from about 0.35 wt. % to about 2.5 wt. %.

Cationic Quaternary Ammonium Compound

According to a preferred embodiment, the antistatic agent for the hair care compositions of the present invention is preferably a quaternary ammonium compound or quaternary ammonium cationic compound, commonly referred to as a quat. Cationic quaternary ammonium compounds can be synthesized by the alkylation of ammonia or other amines. U.S. Pat. No. 7,955,611 discusses the use of cationic quaternary ammonium compounds in hair care products and guidelines for their selection. U.S. Pat. No. 7,955,611 at col. 4, line 55-col. 5, lines 67 is incorporated as if fully set forth herein.

Exemplary quaternary ammonium compounds that may be used as antistatic agents include, for example, alkylated quaternary ammonium compounds, ring or cyclic quaternary ammonium compounds, aromatic quaternary ammonium compounds, diquaternary ammonium compounds, alkoxylated quaternary ammonium compounds, amidoamine quaternary ammonium compounds, ester quaternary ammonium compounds, and mixtures thereof.

Various exemplary quaternary ammonium compounds useful as antistatic agents are described herein. For example, exemplary alkylated quaternary ammonium compounds include ammonium compounds having an alkyl group containing between C₆-C₂₄. Exemplary alkylated quaternary ammonium compounds include monoalkyl trimethyl quaternary ammonium compounds, monomethyl trialkyl quaternary ammonium compounds, and dialkyl dimethyl quaternary ammonium compounds. The alkyl group can be a C₈-C₂₂ group or a C₈-C₁₈ group or a C₁₂-C₂₂ group that is aliphatic and saturated or unsaturated or straight or branched, an alkyl group, a benzyl group, an alkyl ether propyl group, hydrogenated-tallow group, coco group, stearyl group, palmityl group, and soya group. Further, exemplary ring or cyclic quaternary ammonium compounds include imidazolinium quaternary ammonium compounds, such as methyl-1hydr. tallow amido ethyl-2-hydr. tallow imidazolinium-methyl sulfate, methyl-1-tallow amido ethyl-2-tallow imidazolinium-methyl sulfate, methyl-1-oleyl amido ethyl-2-oleyl imidazolinium-methyl sulfate, and 1-ethylene bis(2-tallow, 1-methyl, imidazolinium-methyl sulfate). Still further, exemplary aromatic quaternary ammonium compounds include those compounds that have at least one benzene ring in the structure. Exemplary aromatic quaternary ammonium compounds include dimethyl alkyl benzyl quaternary ammonium compounds, monomethyl dialkyl benzyl quaternary ammonium compounds, trimethyl benzyl quaternary ammonium compounds, and trialkyl benzyl quaternary ammonium compounds. Further, the alkyl group can contain between about 6 and about 24 carbon atoms, and can contain between about 10 and about 18 carbon atoms, and can be a stearyl group or a hydrogenated tallow group. Aromatic quaternary ammonium compounds can include multiple benzyl groups. Exemplary alkoxylated quaternary ammonium compounds include methyldialkoxy alkyl quaternary ammonium compounds, trialkoxy alkyl quaternary ammonium compounds, trialkoxy methyl quaternary ammonium compounds, dimethyl alkoxy alkyl quaternary ammonium compounds, and trimethyl alkoxy quaternary ammonium compounds. The alkyl group can contain between about C₆-C₂₄ and the alkoxy groups can contain between about 1 and about 50 alkoxy groups units wherein each alkoxy unit contains between about C₂-C₃. Exemplary amidoamine quaternary ammonium compounds may include methyl-bis(tallow amidoethyl)-2-hydroxyethyl ammonium methyl sulfate, methyl bis(oleylamidoethyl)-2-hydroxyethyl ammonium methyl sulfate, and methyl bis(hydrtallowamidoethyl)-2-hydroxyethyl ammonium methyl sulfate.

According to a further embodiment, the quaternary ammonium compound utilized as the antistatic agent according to the invention may include any counter ion. Exemplary counter ions include chloride, methyl sulfate, ethyl sulfate, and sulfate. Exemplary cationic quats including a counter ion include behentrimonium chloride and behentrimonium methosulfate, and mixtures thereof. Thus, in an embodiment of the invention, the antistatic agent may comprise a quaternary ammonium compound selected from the group consisting of behentrimonium chloride and behentrimonium methosulfate, and mixtures thereof. One skilled in the art may further prepare quaternary ammonium cation antistatic agents, such as those described herein, through esterification and quaternization reactions, using commercially-available materials, for example as disclosed in U.S. Pat. No. 4,137,180. For example, ester quats according to preferred embodiments of the invention may be obtained by quaternizing fatty triethanolamine esters. See U.S. Pat. No. 6,037,315.

Aqueous Carrier

The compositions of the present invention comprise an aqueous carrier. In some embodiments of the invention, the compositions are in the form of pourable liquids under ambient conditions. As such, certain embodiments comprise an aqueous carrier present in the compositions preferably from about 35 wt. % to about 97.25 wt. %, more preferably from about 40 wt. % to about 90 wt. %, and most preferably from about 42.5 wt. % to about 85 wt. % by weight of the compositions. Moreover, in embodiments of the present invention, the amount of aqueous carrier per the total mass of the composition of the invention may be preferably from about 340 g/kg to about 1025 g/kg, more preferably from about 385 g/kg to about 945 g/kg, and most preferably from about 420 g/kg to about 880 g/kg. The aqueous carrier may comprise water, or a miscible mixture of water and organic solvent. The aqueous carrier may also comprise water substantially free of organic solvents.

Chelating Agent

The compositions of the present invention may optionally include one or more chelating agents. Suitable chelating agents for the compositions of the present invention include, but are not limited to ethylenediaminetetraacetic acid (EDTA) and salts thereof, particularly tetrasodium EDTA and tetrapotassium EDTA, diethylenetriaminepentaacetic acid and salts thereof, and hydroxyethanediphosphonic acid (HEDP) and salts thereof, and mixtures thereof. When used in the compositions of the invention, chelating agent is present preferably from about 0 wt. % to about 0.2 wt. %, more preferably from about 0.05 wt. % to about 0.15 wt. %, and most preferably from about 0.07 wt. % to about 0.1 wt. %.

Conditioning Agent

Compositions of the present invention include at least one conditioning agent. Any known conditioning agent is useful in the personal care compositions of this invention. Conditioning agents function to improve the cosmetic properties of the hair, particularly softness, thickening, untangling, feel, and static electricity and may be in liquid, semi-solid, or solid form such as oils, waxes, or gums. Similarly, any known hair or skin altering agent is useful in the compositions of this invention. Preferred conditioning agents include fatty amines. Mixtures of two or more conditioning agents can be used.

The conditioning agent can be any fatty amine known to be useful as a conditioning agent, e.g., dodecyl, cetyl or stearyl amines, such as stearamidopropyl dimethylamine.

Conditioning agents may be chosen from synthesis oils, mineral oils, vegetable oils, fluorinated or perfluorinated oils, natural or synthetic waxes, silicones, cationic polymers, proteins and hydrolyzed proteins, ceramide type compounds, cationic surfactants, fatty amines, fatty acids and their derivatives, as well as mixtures of these different compounds.

The synthesis oils include polyolefins, e.g., poly-α-olefins such as polybutenes, polyisobutenes and polydecenes. The polyolefins can be hydrogenated.

The mineral oils suitable for use in the compositions of the invention include hexadecane and oil of paraffin.

A list of suitable vegetable oils comprises argan, avocado, corn, glycerol tricaprocaprylate, grapeseed, jojoba, macadamia nut, Purcellin oil, raisin seed, sesame seed, soy, squash, sunflower, walnut oils, and blends thereof.

Suitable natural or synthetic oils include eucalyptus, lavender, vetiver, litsea cubeba, lemon, sandalwood, rosemary, chamomile, savory, nutmeg, cinnamon, hyssop, caraway, orange, geranium, cade, and bergamot.

Suitable natural and synthetic waxes include carnauba wax, candelila wax, alfa wax, paraffin wax, ozokerite wax, vegetable waxes such as olive wax, rice wax, hydrogenated jojoba wax, absolute flower waxes such as black currant flower wax, animal waxes such as bees wax, modified bees wax (cerabellina), marine waxes and polyolefin waxes such as polyethylene wax, and blends thereof.

The cationic polymers that may be used as a conditioning agent according to the invention are those known to improve the cosmetic properties of hair treated by detergent compositions. The expression “cationic polymer” as used herein, indicates any polymer containing cationic groups and/or ionizable groups in cationic groups. The cationic polymers used generally have a molecular weight the average number of which falls between about 500 Daltons and about 5,000,000 Daltons, and preferably between about 1000 Daltons and about 3,000,000 Daltons. The cationic polymer may be chosen from among those containing units including primary, secondary, tertiary, and/or quaternary amine groups that may either form part of the main polymer chain or a side chain.

Useful cationic polymers include known polyamine, polyaminoamide, and quaternary polyammonium types of polymers, such as: (1) Homopolymers and copolymers derived from acrylic or methacrylic esters or amides. The copolymers can contain one or more units derived from acrylamides, methacrylamides, diacetone acrylamides, acrylamides and methacrylamides, acrylic or methacrylic acids or their esters, vinyllactams such as vinyl pyrrolidone or vinyl caprolactam, and vinyl esters. Specific examples include: copolymers of acrylamide and dimethyl amino ethyl methacrylate quaternized with dimethyl sulfate or with an alkyl halide; copolymers of acrylamide and methacryloyl oxyethyl trimethyl ammonium chloride; the copolymer of acrylamide and methacryloyl oxyethyl trimethyl ammonium methosulfate; copolymers of vinyl pyrrolidone/dialkylaminoalkyl acrylate or methacrylate, optionally quaternized, such as the products sold under the name Gafquat™ by International Specialty Products; the dimethyl amino ethyl methacrylate/vinyl caprolactam/vinyl pyrrolidone terpolymers, such as the product sold under the name Gaffix™ VC 713 by International Specialty Products; the vinyl pyrrolidone/methacrylamidopropyl dimethylamine copolymer, marketed under the name Styleze™ CC 10 by International Specialty Products; the vinyl pyrrolidone/quaternized dimethyl amino propyl methacrylamide copolymers such as the product sold under the name Gafquat™ HS 100 by International Specialty Products (Wayne, N.J.), and the terpolymer of N-vinyl-2-pyrrolidone, dimethylaminopropyl methacrylamide, and methacryloylaminopropyl lauryl dimethylammonium chloride, sold under the name Styleze™ W, also by International Specialty Products. (2) Derivatives of cellulose ethers containing quaternary ammonium groups, such as hydroxy ethyl cellulose quaternary ammonium that has reacted with an epoxide substituted by a trimethyl ammonium group. (3) Derivatives of cationic cellulose such as cellulose copolymers or derivatives of cellulose grafted with a hydrosoluble quaternary ammonium monomer, as described in U.S. Pat. No. 4,131,576, such as the hydroxy alkyl cellulose, and the hydroxymethyl-, hydroxyethyl- or hydroxypropyl-cellulose grafted with a salt of methacryloyl ethyl trimethyl ammonium, methacrylamidopropyl trimethyl ammonium, or dimethyl diallyl ammonium. (4) Cationic polysaccharides such as described in U.S. Pat. Nos. 3,589,578 and 4,031,307, guar gums containing cationic trialkyl ammonium groups and guar gums modified by a salt, e.g., chloride of 2,3-epoxy propyl trimethyl ammonium. (5) Polymers composed of piperazinyl units and alkylene or hydroxy alkylene divalent radicals with straight or branched chains, possibly interrupted by atoms of oxygen, sulfur, nitrogen, or by aromatic or heterocyclic cycles, as well as the products of the oxidation and/or quaternization of such polymers. (6) Water-soluble polyamino amides prepared by polycondensation of an acid compound with a polyamine. These polyamino amides may be reticulated. (7) Derivatives of polyamino amides resulting from the condensation of polyalcoylene polyamines with polycarboxylic acids followed by alcoylation by bi-functional agents. (8) Polymers obtained by reaction of a polyalkylene polyamine containing two primary amine groups and at least one secondary amine group with a dioxycarboxylic acid chosen from among diglycolic acid and saturated dicarboxylic aliphatic acids having 3 to 8 atoms of carbon. Such polymers are described in U.S. Pat. Nos. 3,227,615 and 2,961,347. (9) Cyclopolymers of alkyl dialyl amine or dialkyl diallyl ammonium such as the homopolymer of dimethyl diallyl ammonium chloride and copolymers of diallyl dimethyl ammonium chloride and acrylamide. (10) Quaternary diammonium polymers such as hexadimethrine chloride. (11) Quaternary polyammonium polymers, including, for example, Mirapol™ A 15, Mirapol™ AD1, Mirapol™ AZ1, and Mirapol™ 175 products sold by Miranol. (12) Quaternary polymers of vinyl pyrrolidone and vinyl imidazole such as the products sold under the names Luviquat™ FC 905, FC 550, and FC 370 by BASF Corporation. (13) Quaternary polyamines. (14) Reticulated polymers known in the art.

Other cationic polymers that may be used within the context of the invention are cationic proteins or hydrolyzed cationic proteins, polyalkyleneimines such as polyethyleneimines, polymers containing vinyl pyridine or vinyl pyridinium units, condensates of polyamines and epichlorhydrins, quaternary polyurethanes, and derivatives of chitin.

Other cationic polymers are derivatives of quaternary cellulose ethers, the homopolymers and copolymers of dimethyl diallyl ammonium chloride, quaternary polymers of vinyl pyrrolidone and vinyl imidazole, and mixtures thereof.

The conditioning agent can be a protein or hydrolyzed cationic or non-cationic protein. Examples of these compounds include hydrolyzed collagens having triethyl ammonium groups, hydrolyzed collagens having trimethyl ammonium and trimethyl stearyl ammonium chloride groups, hydrolyzed animal proteins having trimethyl benzyl ammonium groups (benzyltrimonium hydrolyzed animal protein), hydrolyzed proteins having groups of quaternary ammonium on the polypeptide chain, including at least one C1-C18 alkyl.

Hydrolyzed proteins include Croquat L, in which the quaternary ammonium groups include a C12 alkyl group, Croquat M, in which the quaternary ammonium groups include C10-C18 alkyl groups, Croquat S in which the quaternary ammonium groups include a C18 alkyl group and Crotein Q in which the quaternary ammonium groups include at least one C1-C18 alkyl group. These products are sold by Croda.

The conditioning agent can comprise quaternized vegetable proteins such as wheat, corn, or soy proteins such as cocodimonium hydrolyzed wheat protein, laurdimonium hydrolyzed wheat protein and steardimonium hydrolyzed wheat protein, 2-N-stearoyl amino-octadecane-1,3-diol, 2-N-behenoyl amino-octadecane-1,3-diol, 2-N-[2-hydroxy-palmitoyl]-amino-octadecane-1,3-diol, 2-N-stearoyl amino-octadecane-1,3,4-triol, N-stearoyl phytosphingosine, 2-N-palmitoyl amino-hexadecane-1,3-diol, bis-(N-hydroxy ethyl N-cetyl)malonamide, N-(2-hydroxy ethyl)-N-(3-cetoxyl-2-hydroxy propyl)amide of cetylic acid, N-docosanoyl N-methyl-D-glucamine and mixtures of such compounds.

The conditioning agent can be a cationic surfactant such as a salt of a primary, secondary, or tertiary fatty amine, optionally polyoxyalkylenated, a quaternary ammonium salt, a derivative of imadazoline, or an amine oxide. Suitable examples include mono-, di-, or tri-alkyl quaternary ammonium compounds with a counterion such as a chloride, methosulfate, tosylate, etc. including, but not limited to, cetrimonium chloride, dicetyldimonium chloride, behentrimonium methosulfate, and the like. The presence of a quaternary ammonium compound in conjunction with the polymer described above reduces static and enhances combing of hair in the dry state. The polymer also enhances the deposition of the quaternary ammonium compound onto the hair substrate thus enhancing the conditioning effect of hair.

The conditioning agent can be a fatty acid or derivatives thereof known to be useful as conditioning agents. Suitable fatty acids include myristic acid, palmitic acid, stearic acid, behenic acid, oleic acid, linoleic acid, and isostearic acid. The derivatives of fatty acids include carboxylic ester acids including mono-, di-, tri- and tetra-carboxylic acids.

The conditioning agent can be a fluorinated or perfluorinated oil. The fluoridated oils may also be fluorocarbons such as fluoramines, e.g., perfluorotributylamine, fluoridated hydrocarbons, such as perfluorodecahydronaphthalene, fluoroesters, and fluoroethers.

The conditioning agent or agents can be present in the composition preferably between about 0.001 wt. % and about 20 wt. %, more preferably between about 0.01 wt. % and about 10 wt. %, and most preferably between about 0.1 wt. % and about 3 wt. %.

Emulsifiers

The compositions of the present invention may optionally include one or more emulsifiers. Suitable emulsifiers for use in the compositions of the invention may include, but are not limited to, ethoxylated fatty alcohols, ethoxylated hydrogenated castor oil, monoesters, polyoxyethylene derivatives of monoesters, mixtures of monoesters and diesters glycerol, and combinations of the same.

Suitable ethoxylated fatty alcohol can be a straight-chain or branched, saturated or unsaturated with a fatty alcohol carbon chain between about eight carbons and about twenty-two carbons. In certain embodiments, a fatty alcohol chain length is about C10-C20 or about C13-C15, and has a degree of ethoxylation of about 1 to about 18, about 5 to about 16, about 7 to about 14, or about 8 to about 12. Non-limiting examples according to the invention include deceth-5, deceth-7, undeceth-7, laureth-4, laureth-5, laureth-6, laureth-7, laureth-8, laureth-9, laureth-10, laureth-12, trideceth-5, trideceth-7, trideceth-8, trideceth-9, trideceth-10, trideceth-12, myreth-8, myreth-10 and myreth-12. Laureth-7, laureth-8, laureth-9, laureth-10, trideceth-5, trideceth-7, trideceth-8, trideceth-9 and trideceth-10.

Suitable ethoxylated hydrogenated castor oils have a degree of ethoxylation of about 5 to about 80, about 10 to about 60, about 25 to about 50, or about 35 to about 45. PEG-10 hydrogenated castor oil, PEG-25 hydrogenated castor oil, PEG-40 hydrogenated castor oil and PEG-60 hydrogenated castor oil are suitable, with PEG-40 hydrogenated castor oil being preferred in certain embodiments.

Suitable monoester and/or a mixture of monoesters and diesters of glycerol can be branched or straight-chain, saturated or unsaturated fatty acids. In certain embodiments their carbon chain length is between about 8-24, about 10-18, about 12-16, with a degree of ethoxylation of about 1-20, about 2-17, about 4-13, or about 6-10. Non-limiting examples include, ethoxylated glyceryl oleates and glyceryl cocoates.

Suitable polyoxyethylene derivatives of monoesters have a degree of ethoxylation of about 5 to about 40, about 10 to about 30, or about 15 to about 25. Moreover, suitable polyoxyethylene derivatives of monoesters are based on monoesters that can be branched or straight-chain, saturated or unsaturated, with a carbon chain length between about 10 and 100. Non-limiting examples of the monoester include, but are not limited to, monolaurate (20 carbons), monopalmitate (40 carbons), monostearate (60 carbons), and monooleate (80 carbons). Suitable polyoxyethylene derivatives of monoesters include, but are not limited to, polysorbate 20, polysorbate 40, polysorbate 60, polysorbate 80, and combinations thereof.

When included in the compositions of present invention, the emulsifier may be present in the composition preferably from about 0 wt. % to about 5 wt. %, more preferably from about 0.05 wt. % to about 4 wt. %, even more preferably from about 0.1 wt. % to about 3.25 wt. %, and most preferably from about 0.4 wt. % to about 2.7 wt. %.

Fats, Waxes and Butters

Some embodiments of the invention may optionally include fats, waxes, and butters. Suitable fats can be solid or liquid vegetable or animal products which consist essentially of mixed glycerol esters of higher fatty acids. Suitable waxes are natural, syntethic, chemically modified, and/or pearlescent waxes. Suitable butters those that are naturally-derived, synthetic, and/or chemically modified. Fats, waxes and butters may be used in any combination or mixture. When included in the compositions of present invention, fats, waxes, and butters may be present in the composition preferably from about 0 wt. % to about 5 wt. %, more preferably from about 0.05 wt. % to about 4 wt. %, even more preferably from about 0.1 wt. % to about 3 wt. %, and most preferably from about 0.3 wt. % to about 1.5 wt. %.

Fats are typically comprised of glycerides and may include additives or fat-like substances, such as lecithins and phospholipids. The term “lecithins” is understood by the person skilled in the art as meaning those glycerophospholipids which are formed from fatty acids, glycerol, phosphoric acid and choline by esterification. Lecithins are therefore also often as phosphatidylcholines in the specialist field. Examples of natural lecithins include kephalins, which are also referred to as phosphatidic acids and are derivatives of 1,2-diacyl-sn-glycerol-3-phosphoric acids. By contrast, phospholipids are usually understood as meaning mono- and preferably diesters of phosphoric acid with glycerol (glycerol phosphates), which are generally included among the fats. In addition, sphingosines and/or sphingolipids are also suitable.

Natural waxes, include, for example, candelilla wax, carnauba wax, Japan wax, espartograss wax, cork wax, guaruma wax, rice germ oil wax, sugarcane wax, ouricury wax, montan wax, beeswax, shellac wax, spermaceti, lanolin (wool wax), uropygeal grease, ceresin, ozokerite (earth wax), petrolatum, paraffin waxes, microwaxes. Synthetic waxes include, for example, synthetic waxes, polyalkylene waxes and polyethylene glycol waxes. Chemically modified waxes (hard waxes), include, for example, montan ester waxes, sasol waxes, and hydrogenated jojoba waxes.

Pearlescent waxes include, for example, alkylene glycol esters, specifically ethylene glycol distearate; fatty acid alkanolamides, specifically coconut fatty acid diethanolamide; partial glycerides, specifically stearic acid monoglyceride; esters of polybasic, optionally hydroxy-substituted carboxylic acids with fatty alcohols having 6 to 22 carbon atoms, specifically long-chain esters of tartaric acid; fatty substances, such as, for example, fatty alcohols, fatty ketones, fatty aldehydes, fatty ethers and fatty carbonates, which have in total at least 24 carbon atoms, specifically laurone and distearyl ether; fatty acids such as stearic acid, hydroxystearic acid or behenic acid, ring-opening products of olefin epoxides having 12 to 22 carbon atoms with fatty alcohols having 12 to 22 carbon atoms and/or polyols having 2 to 15 carbon atoms and 2 to 10 hydroxyl groups, and mixtures thereof.

Butters that are suitable for compositions of the present invention include naturally-derived butters, synthetic butters, and chemically modified butters. Non-limiting, examples of suitable butters include, cocoa butter, cupuacu butter, goa butter, mango butter, and shea butter.

Fatty Alcohol

Optionally, the compositions of the present invention can comprise at least one fatty alcohol. In particular embodiments, individual fatty alcohol compounds or combinations of two or more different fatty alcohol compounds may be selected. Fatty alcohols suitable for use in the present invention are those having from about 4 to about 70 carbon atoms, and in some embodiments from about 7 to about 50 carbon atoms, in other embodiments from about 10 to about 30 carbon atoms, in still further embodiments from about 12 to about 20 carbon atoms, and in even yet other embodiments from about 15 to about 18 carbon atoms.

The fatty alcohols may be straight or branched chain alcohols and may be saturated or unsaturated. Suitable fatty alcohols include, but are not limited, to tert-Butyl alcohol, tert-Amyl alcohol, 3-Methyl-3-pentanol, Ethchlorvynol, capryl alcohol, 2-ethyl hexanol, perlargonic alcohol, capric alcohol, undecyl alcohol, lauryl alcohol, tridecyl alcohol, myristyl alcohol, pentadecyl alcohol, cetyl alcohol, palmitoleyl alcohol, heptadecyl alcohol, stearyl alcohol, nonadecyl alcohol, arachidyl alcohol, heneicosyl alcohol, behenyl alcohol, erucyl alcohol, 1-tricosanol, lignoceryl alcohol, ceryl alcohol, 1-heptacosanol, 1-hexacosanol, 1-octacosanol, 1-nonacosanol, 1-triacontanol, C12-20 alcohols, C20-40 alcohols, C30-50 alcohols, C40-60 alcohols, and mixtures thereof. In certain embodiments, the fatty alcohol may be selected from the group consisting of cetyl alcohol, stearyl alcohol, cetearyl alcohol, and combinations thereof.

The fatty alcohols for use in the present invention may be of natural, vegetable, or synthetic origin. When included in the compositions of present invention, the fatty alcohol may be present in the composition from about 0 wt. % to about 15 wt. %, preferably from about 0.05 wt. % to about 10 wt. %, more preferably from about 0.1 wt. % to about 9 wt. %, even more preferably from about 1 wt. % to about 7.5 wt. %, and most preferably from about 2 wt. % to about 6 wt. % by weight of the hair care composition. In embodiments of the present invention, the amount of fatty alcohol per the total mass of the composition of the invention may be from about 20 g/kg to about 100 g/kg, preferably from about 30 g/kg to about 90 g/kg, more preferably from about 40 g/kg to about 80 g/kg, even more preferably from about 50 g/kg to about 70 g/kg, most preferably from about 55 g/kg to about 60 g/kg.

Film Forming Agents

The compositions of the present invention may optionally include film forming agents. Suitable film forming agents include, but are not limited to chitosan, microcrystalline chitosan, quaternized chitosan, polyvinylpyrrolidone, vinylpyrrolidone-vinyl acetate copolymers such as Luviskol® VA 73W sold by BASF, polymers of the acrylic acid series, quaternary cellulose derivatives, collagen, hyaluronic acid and/or salts thereof and similar compounds. When used, the film forming agents can be present in the compositions preferably from about 0 wt. % to about 20 wt. %, more preferably from about 0.05 wt. % to about 15 wt. %, and most preferably from about 0.1 wt. % to about 12 wt. %, by weight of the composition.

Fixatives

The compositions of the present invention may optionally include fixatives. Suitable fixatives include, but are not limited to, polymers and copolymers of acrylates and acrylic acids, including for example, polyacrylates, acrylic acid crosspolymers, polyvinyl acetates, and vinyl acetates and their copolymers and crosspolymers. In particular embodiments, it may be beneficial for the fixative polymer to be polar and water soluble. Examples of suitable fixatives, include, but are not limited to, polyacrylate-14, polyacrylate-32, acrylates crosspolymer-3, AMP-acrylates/allyl methacrylate copolymer, polyacrylate-2 crosspolymer, polyvinylpyrrolidone, vinyl acetate copolymers, polyvinyl formamide, and blends or combinations thereof. A particularly suitable blend is polyvinylpyrrolidone/vinyl acetate copolymer such as Luviskol® VA 73W sold by BASF. When used, the fixatives can be present in the compositions preferably from about 0.05 wt. % to about 20 wt. %, more preferably from about 1 wt. % to about 15 wt. %, most preferably from about 5 wt. % to about 12 wt. %. In another aspect of the invention, the fixatives can be present in the compositions preferably from about 0.05 g/kg to about 20 g/kg, more preferably from about 1 g/kg to about 15 g/kg, and most preferably from about 5 g/kg to about 12 g/kg.

Humectants

The compositions of the present invention may optionally include humectants. Suitable humectants include, but are not limited to, polyhydric alcohols, water soluble alkoxylated nonionic polymers, and mixtures thereof. When used, the humectants can be present in the compositions preferably from about 0 wt. % to about 20 wt. %, more preferably from about 0.05 wt. % to about 15 wt. %, even more preferably from about 0.1 wt. % to about 10 wt. %, and most preferably from about 0.5 wt. % to about 5 wt. %, by weight of the composition.

Hydrotrope

In some embodiments of the invention, hydrotropes can optionally be added to improve flow behavior of the compositions. Examples of suitable hydrotropes, include, but are not limited to, ethanol, isopropyl alcohol, and polyols. Suitable polyols typically have between about 2 and 15 carbon atoms and at least two hydroxyl groups. The polyols can also contain further functional groups, in particular amino groups, and/or be modified with nitrogen.

Nonlimiting examples of suitable hydrotropes include are glycerol; alkylene glycols, including, for example, ethylene glycol, diethylene glycol, propane-1,2-diol, butylene glycol, hexylene glycol, and polyethylene glycols with an average molecular weight of from about 100 to about 1,000 Daltons; technical-grade oligoglycerol mixtures with a degree of self-condensation of from 1.5 to 10, including, for example, technical-grade diglycerol mixtures with a diglycerol content of from 40 to 50% by weight; methylol compounds, including, for example, trimethylolethane, trimethylolpropane, trimethylolbutane, pentaerythritol and dipentaerythritol; lower alkyl glucosides, in particular those having 1 to 8 carbon atoms in the alkyl radical, such as, for example, methyl glucoside and butyl glucoside; sugar alcohols having about 5 to about 12 carbon atoms, such as, for example, sorbitol or mannitol; sugars having about 5 to about 12 carbon atoms, including, for example, glucose or sucrose; amino sugars, including, for example, glucamine; and dialcoholamines, such as diethanolamine or 2-amino-1,3-propanediol.

When used in the compositions of the present invention, hydrotropes can be present from about preferably 0.01 wt. % to about 10 wt. %, more preferably from about 0.5 wt. % to about 8 wt. %, and most preferably from about 0.8 wt. % to about 6 wt. %.

pH Modifier

The compositions of the present invention may optionally include pH modifiers. In certain embodiments of the invention the pH is preferably between about 3.5 and about 10, more preferably between about 4 and about 9, and most preferably between about 4.5 and about 7.5. In some embodiments of the invention, for example, but not limited to, those optimized for shampoos and styling gels, the pH is preferably between about 3.5 and about 7.0, more preferably between about 4.0 and about 6.5, and most preferably between about 4.5 and about 6.0. In further embodiments of the invention, for example, but not limited to, those optimized for cleansing conditioners, the pH is preferably between about 7.0 and about 9.5, more preferably between about 8.0 and about 9.0, most preferably between about 8.2 and about 8.8. In other embodiments of the invention, for example, but not limited to, those optimized for hair spray, the pH is preferably between about 5.0 and about 9.0, more preferably between about 6.0 and about 8.0, most preferably between about 6.5 and about 7.5.

Suitable pH modifiers for use in the compositions of the present invention, include, but are not limited to, bases and organic acids. Particularly suitable organic acids include, α-hydroxy acids, such as include malic acid, citric acid, lactic acid, glycolic acid, and mixtures thereof. Particularly suitable bases include sodium hydroxide, potassium hydroxide, ammonium chloride, and mixtures thereof. The compositions of the present invention may also include combinations of both bases and organic acids. When used in the compositions of the present invention, pH modifiers can be present preferably from about 0.01 wt. % to about 5 wt. %, more preferably from about 0.1 wt. % to 3 wt. %, even more preferably from about 0.8 wt. % to about 2 wt. %, most preferably from about 1 wt. % to about 1.5 wt. %. In other embodiments, the compositions of the invention may be substantially free of pH modifiers, or any particular type of pH modifier, such as free of bases or organic acids. When the compositions are substantially free of pH modifiers, the pH modifiers may be preferably present at less than about 0.5 wt. %, more preferably present at less than about 0.1 wt. %, and most preferably present at less than about 0.01 wt. %.

Proteins

The compositions of the present invention may optionally include proteins, particularly plant-derived proteins. Suitable proteins may be from barley proteins, collagen proteins, keratin proteins, quinoa proteins, rice proteins, silk proteins, soy proteins, wheat proteins, and combinations of the same. In certain embodiments of the invention, rice quat protein is particularly suitable. When proteins are used in the compositions of the present invention, they can be present preferably from about 0 wt. % to about 1 wt. %, more preferably from about 0.01 wt. % to about 0.5 wt. %, and most preferably from about 0.05 wt. % to about 0.2 wt. %.

Silicones

The compositions of the invention may optionally include silicone compounds. Suitable silicone compounds include, but are not limited to, polyorganosiloxanes that are insoluble in the composition, silicones in the form of oils, waxes, resins, or gums; they may be volatile or non-volatile.

The silicones suitable for use according to the invention include. The silicones can be selected from polyalkyl siloxanes, polyaryl siloxanes, polyalkyl aryl siloxanes, silicone gums and resins, and polyorgano siloxanes modified by organofunctional groups. Non-limiting examples include dimethylpolysiloxanes, methylphenylpolysiloxanes, cyclic silicones, and also amine-, amino-, fatty acid-, alcohol-, polyether-, epoxy-, fluorine-, glycoside- and/or alkyl-modified silicone compounds, and mixtures thereof.

Suitable polyalkyl siloxanes include polydimethyl siloxanes with terminal trimethyl silyl groups or terminal dimethyl silanol groups (dimethiconol) and polyalkyl (C1-C20) siloxanes.

Suitable polyalkyl aryl siloxanes include polydimethyl methyl phenyl siloxanes and polydimethyl diphenyl siloxanes, linear or branched.

The silicone gums suitable for use herein include polydiorganosiloxanes preferably having a number-average molecular weight between 200,000 Da and 1,000,000, Da used alone or mixed with a solvent. Examples include polymethyl siloxane, polydimethyl siloxane/methyl vinyl siloxane gums, polydimethyl siloxane/diphenyl siloxane, polydimethyl siloxane/phenyl methyl siloxane and polydimethyl siloxane/diphenyl siloxane/methyl vinyl siloxane.

Suitable silicone resins include silicones with a dimethyl/trimethyl siloxane structure and resins of the trimethyl siloxysilicate type.

The organo-modified silicones suitable for use in the invention include silicones such as those previously defined and containing one or more organofunctional groups attached by means of a hydrocarbon radical and grafted siliconated polymers. Particularly preferred are amino functional silicones.

The silicones may be used in the form of emulsions, nano-emulsions, or microemulsions.

In certain embodiments of the invention, it has been found that cylcomethicones, dimethicones, amodimethicones, and amodimethicone blends are particularly suitable. A particularly suitable amodimethicone blend contains amodimethicone, trideceth-12, and centrimonium chloride, which is available from Dow Corning under the trade name Xiameter® MEM-8194 Emulsion. In an aspect of the invention, the silicone may be selected based on its viscosity. In such embodiments, the viscosity of the silicone may be between about 20 cps and about 2000 cps. In certain embodiments of the invention, the silicone may be selected from the group consisting of amodimethicone blend, cyclomethicone, dimethyl siloxane, and polydimethylsiloxane.

When used in the compositions of the present invention, silicones can be present preferably from about 0.01 wt. % to about 5 wt. %, more preferably from about 0.05 wt. % to about 3 wt. %, and most preferably from about 0.1 wt. % to about 2 wt. %. In other embodiments of the present invention, silicones may be present preferably from about 74.5 wt. % to about 99.9 wt. %, more preferably from about 84.5 wt. % to about 99.6 wt. %; even more preferably from about 92.4 wt. % to about 99.5 wt. %; and most preferably at about 99.4 wt. %.

Superfatting Agent

The compositions of the invention may optionally include superfatting agents. Suitable superfatting agents include, but are not limited to, lanolin, lecithin, polyethoxlyated or acylated lanoin, lecithin derivatives, polyol fatty acid esters, monoglycerides, and fatty acid alkanolamides. When used in the compositions of the present invention, superfatting agents can be present preferably from about 0 wt. % to about 10 wt. %, more preferably from about 0.01 wt. % to about 5 wt. %, and most preferably from about 0.05 wt. % to about 3 wt. %.

Surfactants

In some embodiments, the compositions of the present invention include one or more surfactants. Surfactants suitable for use with the compositions of the present invention include, but are not limited to, anionic surfactants, amphoteric surfactants, cationic surfactants, nonionic surfactants, and zwitterionic surfactants. Surfactants may be useful for as surfactants and also in providing a number of other desired properties in a hair care product. For example, surfactants may assist in forming emulsions and microemulsions, in separating oils from water to improve cleaning and rinsibility, as foaming agents, and for providing additional emulsifying properties. In some embodiments, the compositions of the present invention include preferably about 2 wt. % to about 50 wt. % of surfactant. In other embodiments the compositions of the present invention include more preferably about 2.5 wt. % to about 40% of a surfactant. In still yet other embodiments, the compositions of the present invention include most preferably about 3 wt. % to about 30 wt. % of surfactant.

Nonionic Surfactants

Useful nonionic surfactants are generally characterized by the presence of an organic hydrophobic group and an organic hydrophilic group and are typically produced by the condensation of an organic aliphatic, alkyl aromatic or polyoxyalkylene hydrophobic compound with a hydrophilic alkaline oxide moiety which in common practice is ethylene oxide or a polyhydration product thereof, polyethylene glycol. Practically any hydrophobic compound having a hydroxyl, carboxyl, amino, or amido group with a reactive hydrogen atom can be condensed with ethylene oxide, or its polyhydration adducts, or its mixtures with alkoxylenes such as propylene oxide to form a nonionic surface-active agent. The length of the hydrophilic polyoxyalkylene moiety which is condensed with any particular hydrophobic compound can be readily adjusted to yield a water dispersible or water soluble compound having the desired degree of balance between hydrophilic and hydrophobic properties. Useful nonionic surfactants include:

1. Block polyoxypropylene-polyoxyethylene polymeric compounds based upon propylene glycol, ethylene glycol, glycerol, trimethylolpropane, and ethylenediamine as the initiator reactive hydrogen compound. Examples of polymeric compounds made from a sequential propoxylation and ethoxylation of initiator are commercially available under the trade names Pluronic® and Tetronic® manufactured by BASF Corp. Pluronic® compounds are difunctional (two reactive hydrogens) compounds formed by condensing ethylene oxide with a hydrophobic base formed by the addition of propylene oxide to the two hydroxyl groups of propylene glycol. This hydrophobic portion of the molecule weighs from about 1,000 to about 4,000. Ethylene oxide is then added to sandwich this hydrophobe between hydrophilic groups, controlled by length to constitute from about 10% by weight to about 80% by weight of the final molecule. Tetronic® compounds are tetra-functional block copolymers derived from the sequential addition of propylene oxide and ethylene oxide to ethylenediamine. The molecular weight of the propylene oxide hydrotype ranges from about 500 to about 7,000; and, the hydrophile, ethylene oxide, is added to constitute from about 10% by weight to about 80% by weight of the molecule.

2. Condensation products of one mole of alkyl phenol wherein the alkyl chain, of straight chain or branched chain configuration, or of single or dual alkyl constituent, contains from about 8 to about 18 carbon atoms with from about 3 to about 50 moles of ethylene oxide. The alkyl group can, for example, be represented by diisobutylene, di-amyl, polymerized propylene, iso-octyl, nonyl, and di-nonyl. These surfactants can be polyethylene, polypropylene, and polybutylene oxide condensates of alkyl phenols. Examples of commercial compounds of this chemistry are available on the market under the trade names Igepal® manufactured by Rhone-Poulenc and Triton® manufactured by Union Carbide.

3. Condensation products of one mole of a saturated or unsaturated, straight or branched chain alcohol having from about 6 to about 24 carbon atoms with from about 3 to about 50 moles of ethylene oxide. The alcohol moiety can consist of mixtures of alcohols in the above delineated carbon range or it can consist of an alcohol having a specific number of carbon atoms within this range. Examples of like commercial surfactant are available under the trade names Neodol™ manufactured by Shell Chemical Co. and Alfonic™ manufactured by Vista Chemical Co.

4. Condensation products of one mole of saturated or unsaturated, straight or branched chain carboxylic acid having from about 8 to about 18 carbon atoms with from about 6 to about 50 moles of ethylene oxide. The acid moiety can consist of mixtures of acids in the above defined carbon atoms range or it can consist of an acid having a specific number of carbon atoms within the range. Examples of commercial compounds of this chemistry are available on the market under the trade names Nopalcol™ manufactured by Henkel Corporation and Lipopeg™ manufactured by Lipo Chemicals, Inc.

In addition to ethoxylated carboxylic acids, commonly called polyethylene glycol esters, other alkanoic acid esters formed by reaction with glycerides, glycerin, and polyhydric (saccharide or sorbitan/sorbitol) alcohols have application in this invention for specialized embodiments, particularly indirect food additive applications. All of these ester moieties have one or more reactive hydrogen sites on their molecule which can undergo further acylation or ethylene oxide (alkoxide) addition to control the hydrophilicity of these substances. Care must be exercised when adding these fatty ester or acylated carbohydrates to compositions of the present invention containing amylase and/or lipase enzymes because of potential incompatibility.

Examples of nonionic low foaming surfactants include:

5. Compounds from (1) which are modified, essentially reversed, by adding ethylene oxide to ethylene glycol to provide a hydrophile of designated molecular weight; and, then adding propylene oxide to obtain hydrophobic blocks on the outside (ends) of the molecule. The hydrophobic portion of the molecule weighs from about 1,000 to about 3,100 with the central hydrophile including 10% by weight to about 80% by weight of the final molecule. These reverse Pluronics™ are manufactured by BASF Corporation under the trade name Pluronic™ R surfactants. Likewise, the Tetronic™ R surfactants are produced by BASF Corporation by the sequential addition of ethylene oxide and propylene oxide to ethylenediamine. The hydrophobic portion of the molecule weighs from about 2,100 to about 6,700 with the central hydrophile including 10% by weight to 80% by weight of the final molecule.

6. Compounds from groups (1), (2), (3) and (4) which are modified by “capping” or “end blocking” the terminal hydroxy group or groups (of multi-functional moieties) to reduce foaming by reaction with a small hydrophobic molecule such as propylene oxide, butylene oxide, benzyl chloride; and, short chain fatty acids, alcohols or alkyl halides containing from 1 to about 5 carbon atoms; and mixtures thereof. Also included are reactants such as thionyl chloride which convert terminal hydroxy groups to a chloride group. Such modifications to the terminal hydroxy group may lead to all-block, block-heteric, heteric-block or all-heteric nonionics.

Additional examples of effective low foaming nonionics include:

7. The alkylphenoxypolyethoxyalkanols of U.S. Pat. No. 2,903,486 issued Sep. 8, 1959 to Brown et al. and represented by the formula

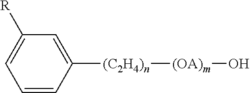

in which R is an alkyl group of 8 to 9 carbon atoms, A is an alkylene chain of 3 to 4 carbon atoms, n is an integer of 7 to 16, and m is an integer of 1 to 10.

The polyalkylene glycol condensates of U.S. Pat. No. 3,048,548 issued Aug. 7, 1962 to Martin et al. having alternating hydrophilic oxyethylene chains and hydrophobic oxypropylene chains where the weight of the terminal hydrophobic chains, the weight of the middle hydrophobic unit and the weight of the linking hydrophilic units each represent about one-third of the condensate.

The defoaming nonionic surfactants disclosed in U.S. Pat. No. 3,382,178 issued May 7, 1968 to Lissant et al. having the general formula Z[(OR)ₙOH]_{z} wherein Z is alkoxylatable material, R is a radical derived from an alkaline oxide which can be ethylene and propylene and n is an integer from, for example, 10 to 2,000 or more and z is an integer determined by the number of reactive oxyalkylatable groups.

The conjugated polyoxyalkylene compounds described in U.S. Pat. No. 2,677,700, issued May 4, 1954 to Jackson et al. corresponding to the formula Y(C₃H₆O)ₙ(C₂H₄O)ₘH wherein Y is the residue of organic compound having from about 1 to 6 carbon atoms and one reactive hydrogen atom, n has an average value of at least about 6.4, as determined by hydroxyl number and m has a value such that the oxyethylene portion constitutes about 10% to about 90% by weight of the molecule.

The conjugated polyoxyalkylene compounds described in U.S. Pat. No. 2,674,619, issued Apr. 6, 1954 to Lundsted et al. having the formula Y[(C₃H₆Oₙ(C₂H₄O)ₘH]ₓ wherein Y is the residue of an organic compound having from about 2 to 6 carbon atoms and containing x reactive hydrogen atoms in which x has a value of at least about 2, n has a value such that the molecular weight of the polyoxypropylene hydrophobic base is at least about 900 and m has value such that the oxyethylene content of the molecule is from about 10% to about 90% by weight. Compounds falling within the scope of the definition for Y include, for example, propylene glycol, glycerine, pentaerythritol, trimethylolpropane, ethylenediamine and the like. The oxypropylene chains optionally, but advantageously, contain small amounts of ethylene oxide and the oxyethylene chains also optionally, but advantageously, contain small amounts of propylene oxide.

Additional conjugated polyoxyalkylene surface-active agents which are advantageously used in the compositions of this invention correspond to the formula: P[(C₃H₆O)ₙ(C₂H₄O)ₘH]ₓ wherein P is the residue of an organic compound having from about 8 to 18 carbon atoms and containing x reactive hydrogen atoms in which x has a value of 1 or 2, n has a value such that the molecular weight of the polyoxyethylene portion is at least about 44 and m has a value such that the oxypropylene content of the molecule is from about 10% to about 90% by weight. In either case the oxypropylene chains may contain optionally, but advantageously, small amounts of ethylene oxide and the oxyethylene chains may contain also optionally, but advantageously, small amounts of propylene oxide.

8. Polyhydroxy fatty acid amide surfactants suitable for use in the present compositions include those having the structural formula R₂CON_{R1}Z in which: R1 is H, C₁-C₄ hydrocarbyl, 2-hydroxy ethyl, 2-hydroxy propyl, ethoxy, propoxy group, or a mixture thereof; R₂ is a C₅-C₃₁ hydrocarbyl, which can be straight-chain; and Z is a polyhydroxyhydrocarbyl having a linear hydrocarbyl chain with at least 3 hydroxyls directly connected to the chain, or an alkoxylated derivative (preferably ethoxylated or propoxylated) thereof. Z can be derived from a reducing sugar in a reductive amination reaction; such as a glycityl moiety.

9. The alkyl ethoxylate condensation products of aliphatic alcohols with from about 0 to about 25 moles of ethylene oxide are suitable for use in the present compositions. The alkyl chain of the aliphatic alcohol can either be straight or branched, primary or secondary, and generally contains from 6 to 22 carbon atoms.

10. The ethoxylated C₆-C₁₈ fatty alcohols and C₆-C₁₈ mixed ethoxylated and propoxylated fatty alcohols are suitable surfactants for use in the present compositions, particularly those that are water soluble. Suitable ethoxylated fatty alcohols include the C₆-C₁₈ ethoxylated fatty alcohols with a degree of ethoxylation of from 3 to 50.

11. Suitable nonionic alkylpolysaccharide surfactants, particularly for use in the present compositions include those disclosed in U.S. Pat. No. 4,565,647, Llenado, issued Jan. 21, 1986. These surfactants include a hydrophobic group containing from about 6 to about 30 carbon atoms and a polysaccharide, e.g., a polyglycoside, hydrophilic group containing from about 1.3 to about 10 saccharide units. Any reducing saccharide containing 5 or 6 carbon atoms can be used, e.g., glucose, galactose and galactosyl moieties can be substituted for the glucosyl moieties. (Optionally the hydrophobic group is attached at the 2-, 3-, 4-, etc. positions thus giving a glucose or galactose as opposed to a glucoside or galactoside.) The intersaccharide bonds can be, e.g., between the one position of the additional saccharide units and the 2-, 3-, 4-, and/or 6-positions on the preceding saccharide units.

12. Fatty acid amide surfactants suitable for use the present compositions include those having the formula: R₆CON(R₇)₂ in which R₆ is an alkyl group containing from 7 to 21 carbon atoms and each R₇ is independently hydrogen, C₁-C₄ alkyl, C₁-C₄ hydroxyalkyl, or —(C₂H₄O)ₓH, where x is in the range of from 1 to 3.

13. A useful class of non-ionic surfactants include the class defined as alkoxylated amines or, most particularly, alcohol alkoxylated/aminated/alkoxylated surfactants. These non-ionic surfactants may be at least in part represented by the general formulae: R²⁰—(PO)_{S}N-(EO)ₜH, R²⁰—(PO)_{S}N-(EO)ₜH(EO)ₜH, and R²⁰—N(EO)ₜH; in which R²⁰ is an alkyl, alkenyl or other aliphatic group, or an alkyl-aryl group of from 8 to 20, preferably 12 to 14 carbon atoms, EO is oxyethylene, PO is oxypropylene, s is 1 to 20, preferably 2-5, t is 1-10, preferably 2-5, and u is 1-10, preferably 2-5. Other variations on the scope of these compounds may be represented by the alternative formula: R²⁰—(PO)_{V}—N[(EO)_{w}H][(EO)_{z}H] in which R²⁰ is as defined above, v is 1 to 20 (e.g., 1, 2, 3, or 4 (preferably 2)), and w and z are independently 1-10, preferably 2-5. These compounds are represented commercially by a line of products sold by Huntsman Chemicals as nonionic surfactants. A preferred chemical of this class includes Surfonic™ PEA 25 Amine Alkoxylate. Preferred nonionic surfactants for the compositions of the invention include alcohol alkoxylates, EO/PO block copolymers, alkylphenol alkoxylates, and the like.

The treatise Nonionic Surfactants, edited by Schick, M. J., Vol. 1 of the Surfactant Science Series, Marcel Dekker, Inc., New York, 1983 is an excellent reference on the wide variety of nonionic compounds generally employed in the practice of the present invention. A typical listing of nonionic classes, and species of these surfactants, is given in U.S. Pat. No. 3,929,678 issued to Laughlin and Heuring on Dec. 30, 1975. Further examples are given in “Surface Active Agents and detergents” (Vol. I and II by Schwartz, Perry and Berch).

Semi-Polar Nonionic Surfactants

The semi-polar type of nonionic surface active agents are another class of nonionic surfactant useful in compositions of the present invention. Generally, semi-polar nonionics are high foamers and foam stabilizers, which can limit their application in CIP systems. However, within compositional embodiments of this invention designed for high foam cleaning methodology, semi-polar nonionics would have immediate utility. The semi-polar nonionic surfactants include the amine oxides, phosphine oxides, sulfoxides and their alkoxylated derivatives.

14. Amine oxides are tertiary amine oxides corresponding to the general formula:

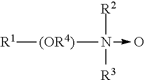

wherein the arrow is a conventional representation of a semi-polar bond; and, R¹, R², and R³ may be aliphatic, aromatic, heterocyclic, alicyclic, or combinations thereof. Generally, for amine oxides useful in hair care products, R¹ is an alkyl radical of from about 8 to about 24 carbon atoms; R² and R³ are alkyl or hydroxyalkyl of 1-3 carbon atoms or a mixture thereof; R² and R³ can be attached to each other, e.g., through an oxygen or nitrogen atom, to form a ring structure; R⁴ is an alkaline or a hydroxyalkylene group containing 2 to 3 carbon atoms; and n ranges from 0 to about 20.

Useful water soluble amine oxide surfactants are selected from the coconut or tallow alkyl di-(lower alkyl) amine oxides, specific examples of which are dodecyldimethylamine oxide, tridecyldimethylamine oxide, etradecyldimethylamine oxide, pentadecyldimethylamine oxide, hexadecyldimethylamine oxide, heptadecyldimethylamine oxide, octadecyldimethylaine oxide, dodecyldipropylamine oxide, tetradecyldipropylamine oxide, hexadecyldipropylamine oxide, tetradecyldibutylamine oxide, octadecyldibutylamine oxide, bis(2-hydroxyethyl)dodecylamine oxide, bis(2-hydroxyethyl)-3-dodecoxy-1-hydroxypropylamine oxide, dimethyl-(2-hydroxydodecyl)amine oxide, 3,6,9-trioctadecyldimethylamine oxide and 3-dodecoxy-2-hydroxypropyldi-(2-hydroxyethyl)amine oxide.

Useful semi-polar nonionic surfactants also include the water soluble phosphine oxides having the following structure:

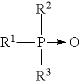

wherein the arrow is a conventional representation of a semi-polar bond; and, R¹ is an alkyl, alkenyl or hydroxyalkyl moiety ranging from 10 to about 24 carbon atoms in chain length; and, R² and R³ are each alkyl moieties separately selected from alkyl or hydroxyalkyl groups containing 1 to 3 carbon atoms.

Examples of useful phosphine oxides include dimethyldecylphosphine oxide, dimethyltetradecylphosphine oxide, methylethyltetradecylphosphone oxide, dimethylhexadecylphosphine oxide, diethyl-2-hydroxyoctyldecylphosphine oxide, bis(2-hydroxyethyl)dodecylphosphine oxide, and bis(hydroxymethyl)tetradecylphosphine oxide.

Semi-polar nonionic surfactants useful herein also include the water soluble sulfoxide compounds which have the structure:

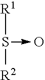

wherein the arrow is a conventional representation of a semi-polar bond; and, R¹ is an alkyl or hydroxyalkyl moiety of about 8 to about 28 carbon atoms, from 0 to about 5 ether linkages and from 0 to about 2 hydroxyl substituents; and R² is an alkyl moiety consisting of alkyl and hydroxyalkyl groups having 1 to 3 carbon atoms.

Useful examples of these sulfoxides include dodecyl methyl sulfoxide; 3-hydroxy tridecyl methyl sulfoxide; 3-methoxy tridecyl methyl sulfoxide; and 3-hydroxy-4-dodecoxybutyl methyl sulfoxide.

Semi-polar nonionic surfactants for the compositions of the invention include dimethyl amine oxides, such as lauryl dimethyl amine oxide, myristyl dimethyl amine oxide, cetyl dimethyl amine oxide, combinations thereof, and the like. Useful water soluble amine oxide surfactants are selected from the octyl, decyl, dodecyl, isododecyl, coconut, or tallow alkyl di-(lower alkyl) amine oxides, specific examples of which are octyldimethylamine oxide, nonyldimethylamine oxide, decyldimethylamine oxide, undecyldimethylamine oxide, dodecyldimethylamine oxide, iso-dodecyldimethyl amine oxide, tridecyldimethylamine oxide, tetradecyldimethylamine oxide, pentadecyldimethylamine oxide, hexadecyldimethylamine oxide, heptadecyldimethylamine oxide, octadecyldimethylaine oxide, dodecyldipropylamine oxide, tetradecyldipropylamine oxide, hexadecyldipropylamine oxide, tetradecyldibutylamine oxide, octadecyldibutylamine oxide, bis(2-hydroxyethyl)dodecylamine oxide, bis(2-hydroxyethyl)-3-dodecoxy-1-hydroxypropylamine oxide, dimethyl-(2-hydroxydodecyl)amine oxide, 3,6,9-trioctadecyldimethylamine oxide and 3-dodecoxy-2-hydroxypropyldi-(2-hydroxyethyl)amine oxide.

Suitable nonionic surfactants suitable for use with the compositions of the present invention include alkoxylated surfactants. Suitable alkoxylated surfactants include EO/PO copolymers, capped EO/PO copolymers, alcohol alkoxylates, capped alcohol alkoxylates, mixtures thereof, or the like. Suitable alkoxylated surfactants for use as solvents include EO/PO block copolymers, such as the Pluronic and reverse Pluronic surfactants; alcohol alkoxylates, such as Dehypon LS-54 (R-(EO)₅(PO)₄) and Dehypon LS-36 (R-(EO)₃(PO)₆); and capped alcohol alkoxylates, such as Plurafac LF221 and Tegoten EC11; mixtures thereof, or the like.

Anionic Surfactants

Also useful in the present invention are surface active substances which are categorized as anionics because the charge on the hydrophobe is negative; or surfactants in which the hydrophobic section of the molecule carries no charge unless the pH is elevated to neutrality or above (e.g. carboxylic acids). Carboxylate, sulfonate, sulfate and phosphate are the polar (hydrophilic) solubilizing groups found in anionic surfactants. Of the cations (counter ions) associated with these polar groups, sodium, lithium and potassium impart water solubility; ammonium and substituted ammonium ions provide both water and oil solubility; and, calcium, barium, and magnesium promote oil solubility. As those skilled in the art understand, anionics are excellent detersive surfactants and therefore can be useful in hair care product compositions.

Anionic sulfate surfactants suitable for use in the present compositions include alkyl ether sulfates, alkyl sulfates, the linear and branched primary and secondary alkyl sulfates, alkyl ethoxysulfates, fatty oleyl glycerol sulfates, alkyl phenol ethylene oxide ether sulfates, the C₅-C₁₇ acyl-N—(C₁-C₄ alkyl) and —N—(C₁-C₂ hydroxyalkyl) glucamine sulfates, and sulfates of alkylpolysaccharides such as the sulfates of alkylpolyglucoside, and the like. Also included are the alkyl sulfates, alkyl poly(ethyleneoxy) ether sulfates and aromatic poly(ethyleneoxy) sulfates such as the sulfates or condensation products of ethylene oxide and nonyl phenol (usually having 1 to 6 oxyethylene groups per molecule).

Anionic sulfonate surfactants suitable for use in the present compositions also include alkyl sulfonates, the linear and branched primary and secondary alkyl sulfonates, and the aromatic sulfonates with or without substituents.

Anionic carboxylate surfactants suitable for use in the present compositions include carboxylic acids (and salts), such as alkanoic acids (and alkanoates), ester carboxylic acids (e.g. alkyl succinates), ether carboxylic acids, sulfonated fatty acids, such as sulfonated oleic acid, and the like. Such carboxylates include alkyl ethoxy carboxylates, alkyl aryl ethoxy carboxylates, alkyl polyethoxy polycarboxylate surfactants and soaps (e.g. alkyl carboxyls). Secondary carboxylates useful in the present compositions include those which contain a carboxyl unit connected to a secondary carbon. The secondary carbon can be in a ring structure, e.g. as in p-octyl benzoic acid, or as in alkyl-substituted cyclohexyl carboxylates. The secondary carboxylate surfactants typically contain no ether linkages, no ester linkages and no hydroxyl groups. Further, they typically lack nitrogen atoms in the head-group (amphiphilic portion). Suitable secondary soap surfactants typically contain 11-13 total carbon atoms, although more carbons atoms (e.g., up to 16) can be present. Suitable carboxylates also include acylamino acids (and salts), such as acylgluamates, acyl peptides, sarcosinates (e.g. N-acyl sarcosinates), taurates (e.g. N-acyl taurates and fatty acid amides of methyl tauride), and the like.

Suitable anionic surfactants include alkyl or alkylaryl ethoxy carboxylates of the following formula:

R—O—(CH₂CH₂O)ₙ(CH₂)ₘ—CO₂X (3)

in which R is a C₈ to C₂₂ alkyl group or

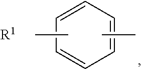

in which R¹ is a C₄-C₁₆ alkyl group; n is an integer of 1-20; m is an integer of 1-3; and X is a counter ion, such as hydrogen, sodium, potassium, lithium, ammonium, or an amine salt such as monoethanolamine (cocamide MEA), diethanolamine or triethanolamine. In some embodiments, n is an integer of 4 to 10 and m is 1. In some embodiments, R is a C₈-C₁₆ alkyl group. In some embodiments, R is a C₁₂-C₁₄ alkyl group, n is 4, and m is 1.

In other embodiments, R is

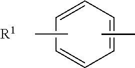

and R¹ is a C₆-C₁₂ alkyl group. In still yet other embodiments, R¹ is a C₉ alkyl group, n is 10 and m is 1.

Such alkyl and alkylaryl ethoxy carboxylates are commercially available. These ethoxy carboxylates are typically available as the acid forms, which can be readily converted to the anionic or salt form. Commercially available carboxylates include, Neodox 23-4, a C₁₂₋₁₃ alkyl polyethoxy (4) carboxylic acid (Shell Chemical), and Emcol CNP-110, a C₉ alkylaryl polyethoxy (10) carboxylic acid (Witco Chemical). Carboxylates are also available from Clariant, e.g. the product Sandopan® DTC, a C₁₃ alkyl polyethoxy (7) carboxylic acid.

Cationic Surfactants

Surface active substances are classified as cationic if the charge on the hydrotrope portion of the molecule is positive. Surfactants in which the hydrotrope carries no charge unless the pH is lowered close to neutrality or lower, but which are then cationic (e.g. alkyl amines), are also included in this group. In theory, cationic surfactants may be synthesized from any combination of elements containing an “onium” structure RnX+Y— and could include compounds other than nitrogen (ammonium) such as phosphorus (phosphonium) and sulfur (sulfonium). In practice, the cationic surfactant field is dominated by nitrogen containing compounds, probably because synthetic routes to nitrogenous cationics are simple and straightforward and give high yields of product, which can make them less expensive.

Cationic surfactants preferably include, more preferably refer to, compounds containing at least one long carbon chain hydrophobic group and at least one positively charged nitrogen. The long carbon chain group may be attached directly to the nitrogen atom by simple substitution; or more preferably indirectly by a bridging functional group or groups in so-called interrupted alkylamines and amido amines. Such functional groups can make the molecule more hydrophilic and/or more water dispersible, more easily water solubilized by co-surfactant mixtures, and/or water soluble. For increased water solubility, additional primary, secondary or tertiary amino groups can be introduced or the amino nitrogen can be quaternized with low molecular weight alkyl groups. Further, the nitrogen can be a part of branched or straight chain moiety of varying degrees of unsaturation or of a saturated or unsaturated heterocyclic ring. In addition, cationic surfactants may contain complex linkages having more than one cationic nitrogen atom.

The surfactant compounds classified as amine oxides, amphoterics and zwitterions are themselves typically cationic in near neutral to acidic pH solutions and can overlap surfactant classifications. Polyoxyethylated cationic surfactants generally behave like nonionic surfactants in alkaline solution and like cationic surfactants in acidic solution.

The simplest cationic amines, amine salts and quaternary ammonium compounds can be schematically drawn thus:

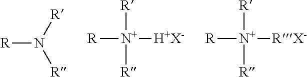

in which, R represents a long alkyl chain, R′, R″, and R′″ may be either long alkyl chains or smaller alkyl or aryl groups or hydrogen and X represents an anion. The amine salts and quaternary ammonium compounds are preferred for practical use in this invention due to their high degree of water solubility.

The majority of large volume commercial cationic surfactants can be subdivided into four major classes and additional sub-groups known to those or skill in the art and described in “Surfactant Encyclopedia”, Cosmetics & Toiletries, Vol. 104 (2) 86-96 (1989). The first class includes alkylamines and their salts. The second class includes alkyl imidazolines. The third class includes ethoxylated amines. The fourth class includes quaternaries, such as alkylbenzyldimethylammonium salts, alkyl benzene salts, heterocyclic ammonium salts, tetra alkylammonium salts, and the like. Cationic surfactants are known to have a variety of properties that can be beneficial in the present compositions. These desirable properties can include detergency in compositions of or below neutral pH, antimicrobial efficacy, thickening or gelling in cooperation with other agents, and the like.

Cationic surfactants useful in the compositions of the present invention include those having the formula R¹ₘR²ₓY_{L}Z wherein each R¹ is an organic group containing a straight or branched alkyl or alkenyl group optionally substituted with up to three phenyl or hydroxy groups and optionally interrupted by up to four of the following structures:

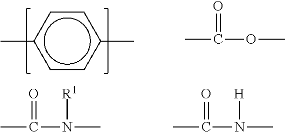

or an isomer or mixture of these structures, and which contains from about 8 to 22 carbon atoms. The R¹ groups can additionally contain up to 12 ethoxy groups. m is a number from 1 to 3. Preferably, no more than one R¹ group in a molecule has 16 or more carbon atoms when m is 2 or more than 12 carbon atoms when m is 3. Each R² is an alkyl or hydroxyalkyl group containing from 1 to 4 carbon atoms or a benzyl group with no more than one R² in a molecule being benzyl, and x is a number from 0 to 11, preferably from 0 to 6. The remainder of any carbon atom positions on the Y group are filled by hydrogens. Y is can be a group including, but not limited to:

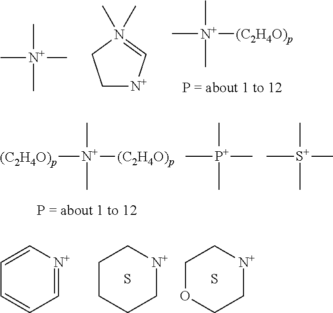

or a mixture thereof. Preferably, L is 1 or 2, with the Y groups being separated by a moiety selected from R¹ and R² analogs (preferably alkylene or alkenylene) having from 1 to about 22 carbon atoms and two free carbon single bonds when L is 2. Z is a water soluble anion, such as a halide, sulfate, methylsulfate, hydroxide, or nitrate anion, particularly preferred being chloride, bromide, iodide, sulfate or methyl sulfate anions, in a number to give electrical neutrality of the cationic component.

Amphoteric Surfactants

Amphoteric, or ampholytic, surfactants contain both a basic and an acidic hydrophilic group and an organic hydrophobic group. These ionic entities may be any of anionic or cationic groups described herein for other types of surfactants. A basic nitrogen and an acidic carboxylate group are the typical functional groups employed as the basic and acidic hydrophilic groups. In a few surfactants, sulfonate, sulfate, phosphonate or phosphate provide the negative charge.

Amphoteric surfactants can be broadly described as derivatives of aliphatic secondary and tertiary amines, in which the aliphatic radical may be straight chain or branched and wherein one of the aliphatic substituents contains from about 8 to 18 carbon atoms and one contains an anionic water solubilizing group, e.g., carboxy, sulfo, sulfato, phosphato, or phosphono. Amphoteric surfactants are subdivided into two major classes known to those of skill in the art and described in “Surfactant Encyclopedia” Cosmetics & Toiletries, Vol. 104 (2) 69-71 (1989), which is herein incorporated by reference in its entirety. The first class includes acyl/dialkyl ethylenediamine derivatives (e.g. 2-alkyl hydroxyethyl imidazoline derivatives) and their salts. The second class includes N-alkylamino acids and their salts. Some amphoteric surfactants can be envisioned as fitting into both classes.

Amphoteric surfactants can be synthesized by methods known to those of skill in the art. For example, 2-alkyl hydroxyethyl imidazoline is synthesized by condensation and ring closure of a long chain carboxylic acid (or a derivative) with dialkyl ethylenediamine. Commercial amphoteric surfactants are derivatized by subsequent hydrolysis and ring-opening of the imidazoline ring by alkylation—for example with chloroacetic acid or ethyl acetate. During alkylation, one or two carboxy-alkyl groups react to form a tertiary amine and an ether linkage with differing alkylating agents yielding different tertiary amines.

Long chain imidazole derivatives having application in the present invention generally have the general formula:

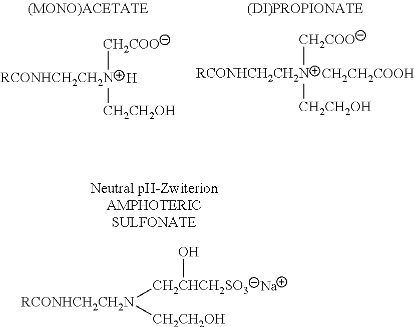

wherein R is an acyclic hydrophobic group containing from about 8 to 18 carbon atoms and M is a cation to neutralize the charge of the anion, generally sodium. Commercially prominent imidazoline-derived amphoterics that can be employed in the present compositions include for example: Cocoamphopropionate, Cocoamphocarboxy-propionate, Cocoamphoglycinate, Cocoamphocarboxy-glycinate, Cocoamphopropyl-sulfonate, and Cocoamphocarboxy-propionic acid. Amphocarboxylic acids can be produced from fatty imidazolines in which the dicarboxylic acid functionality of the amphodicarboxylic acid is diacetic acid and/or dipropionic acid.

The carboxymethylated compounds (glycinates) described herein above frequently are called betaines. Betaines are a special class of amphoteric discussed herein below in the section entitled, Zwitterion Surfactants.

Long chain N-alkylamino acids are readily prepared by reaction RNH₂, in which R=C₈-C₁₈ straight or branched chain alkyl, fatty amines with halogenated carboxylic acids. Alkylation of the primary amino groups of an amino acid leads to secondary and tertiary amines. Alkyl substituents may have additional amino groups that provide more than one reactive nitrogen center. Most commercial N-alkylamine acids are alkyl derivatives of beta-alanine or beta-N(2-carboxyethyl) alanine Examples of commercial N-alkylamino acid ampholytes having application in this invention include alkyl beta-amino dipropionates, RN(C₂H₄COOM)₂ and RNHC₂H₄COOM. In an embodiment, R can be an acyclic hydrophobic group containing from about 8 to about 18 carbon atoms, and M is a cation to neutralize the charge of the anion.

Suitable amphoteric surfactants include those derived from coconut products such as coconut oil or coconut fatty acid, including sodium cocoyl isethionate. Additional suitable coconut derived surfactants include as part of their structure an ethylenediamine moiety, an alkanolamide moiety, an amino acid moiety, e.g., glycine, or a combination thereof; and an aliphatic substituent of from about 8 to 18 (e.g., 12) carbon atoms. Such a surfactant can also be considered an alkyl amphodicarboxylic acid. These amphoteric surfactants can include chemical structures represented as: C₁₂-alkyl-C(O)—NH—CH₂—CH₂—N⁺(CH₂—CH₂—CO₂Na)₂—CH₂—CH₂—OH or C₁₂-alkyl-C(O)—N(H)—CH₂—CH₂—N⁺(CH₂—CO₂Na)₂-CH₂—CH₂—OH. Disodium cocoampho dipropionate is one suitable amphoteric surfactant and is commercially available under the tradename Miranol™ FBS from Rhodia Inc., Cranbury, N.J. Another suitable coconut derived amphoteric surfactant with the chemical name disodium cocoampho diacetate is sold under the tradename Mirataine™ JCHA, also from Rhodia Inc., Cranbury, N.J.

A typical listing of amphoteric classes, and species of these surfactants, is given in U.S. Pat. No. 3,929,678 issued to Laughlin and Heuring on Dec. 30, 1975. Further examples are given in “Surface Active Agents and Detergents” (Vol. I and II by Schwartz, Perry and Berch). Each of these references are herein incorporated by reference in their entirety.

Zwitterionic Surfactants

Zwitterionic surfactants can be thought of as a subset of the amphoteric surfactants and can include an anionic charge. Zwitterionic surfactants can be broadly described as derivatives of secondary and tertiary amines, derivatives of heterocyclic secondary and tertiary amines, or derivatives of quaternary ammonium, quaternary phosphonium or tertiary sulfonium compounds. Typically, a zwitterionic surfactant includes a positive charged quaternary ammonium or, in some cases, a sulfonium or phosphonium ion; a negative charged carboxyl group; and an alkyl group. Zwitterionics generally contain cationic and anionic groups which ionize to a nearly equal degree in the isoelectric region of the molecule and which can develop strong “inner-salt” attraction between positive-negative charge centers. Examples of such zwitterionic synthetic surfactants include derivatives of aliphatic quaternary ammonium, phosphonium, and sulfonium compounds, in which the aliphatic radicals can be straight chain or branched, and wherein one of the aliphatic substituents contains from 8 to 18 carbon atoms and one contains an anionic water solubilizing group, e.g., carboxy, sulfonate, sulfate, phosphate, or phosphonate.

Betaine and sultaine surfactants are exemplary zwitterionic surfactants for use herein. A general formula for these compounds is:

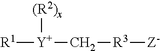

wherein R¹ contains an alkyl, alkenyl, or hydroxyalkyl radical of from 8 to 18 carbon atoms having from 0 to 10 ethylene oxide moieties and from 0 to 1 glyceryl moiety; Y is selected from the group consisting of nitrogen, phosphorus, and sulfur atoms; R² is an alkyl or monohydroxy alkyl group containing 1 to 3 carbon atoms; x is 1 when Y is a sulfur atom and 2 when Y is a nitrogen or phosphorus atom, R³ is an alkylene or hydroxy alkylene or hydroxy alkylene of from 1 to 4 carbon atoms and Z is a radical selected from the group consisting of carboxylate, sulfonate, sulfate, phosphonate, and phosphate groups.

Examples of zwitterionic surfactants having the structures listed above include: 4-[N,N-di(2-hydroxyethyl)-N-octadecylammonio]-butane-1-carboxylate; 5-[S-3-hydroxypropyl-S-hexadecylsulfonio]-3-hydroxypentane-1-sulfate; 3-[P,P-diethyl-P-3,6,9-trioxatetracosanephosphonio]-2-hydroxypropane-1-phosphate; 3-[N,N-dipropyl-N-3-dodecoxy-2-hydroxypropyl-ammonio]-propane-1-phosphonate; 3-(N,N-dimethyl-N-hexadecylammonio)-propane-1-sulfonate; 3-(N,N-dimethyl-N-hexadecylammonio)-2-hydroxy-propane-1-sulfonate; 4-[N,N-di(2(2-hydroxyethyl)-N(2-hydroxydodecyl)ammonio]-butane-1-carboxylate; 3-[S-ethyl-S-(3-dodecoxy-2-hydroxypropyl)sulfonio]-propane-1-phosphate; 3-[P,P-dimethyl-P-dodecylphosphonio]-propane-1-phosphonate; and S[N,N-di(3-hydroxypropyl)-N-hexadecylammonio]-2-hydroxy-pentane-1-sulfate. The alkyl groups contained in said surfactants can be straight or branched and saturated or unsaturated.

The zwitterionic surfactant suitable for use in the present compositions includes a betaine of the general structure:

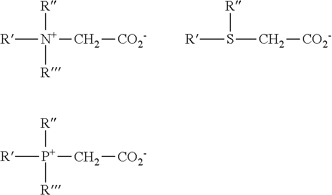

These surfactant betaines typically do not exhibit strong cationic or anionic characters at pH extremes nor do they show reduced water solubility in their isoelectric range. Unlike “external” quaternary ammonium salts, betaines are compatible with anionics. Examples of suitable betaines include coconut acylamidopropyldimethyl betaine; cocamidopropyl betaine; hexadecyl dimethyl betaine; C₁₂₋₁₄ acylamidopropylbetaine; C₈₋₁₄ acylamidohexyldiethyl betaine; 4-C₁₄₋₁₆ acylmethylamidodiethylammonio-1-carboxybutane; C₁₆₋₁₈ acylamidodimethylbetaine; C₁₂₋₁₆ acylamidopentanediethylbetaine; and C₁₂₋₁₆ acylmethylamidodimethylbetaine.

Sultaines useful in the present invention include those compounds having the formula (R(R¹)₂N⁺R²SO³⁻, in which R is a C₆-C₁₈ hydrocarbyl group, each R¹ is typically independently C₁-C₃ alkyl, e.g. methyl, and R² is a C₁-C₆ hydrocarbyl group, e.g. a C₁-C₃ alkylene or hydroxyalkylene group.

A typical listing of zwitterionic classes, and species of these surfactants, is given in U.S. Pat. No. 3,929,678 issued to Laughlin and Heuring on Dec. 30, 1975. Further examples are given in “Surface Active Agents and Detergents” (Vol. I and II by Schwartz, Perry and Berch).

Suspending Agent

The compositions of the present invention may optionally comprise a suspending agent at concentrations effective for suspending water-insoluble material in dispersed form in the compositions or for modifying the viscosity of the composition. Suitable suspending agents include, but are not limited to, crystalline suspending agents such as acyl derivatives, long chain amine oxides, and mixtures thereof and are described in U.S. Pat. No. 4,741,855. These suspending agents may include ethylene glycol esters of fatty acids having from about 16 to about 22 carbon atoms. Alternatives include ethylene glycol stearates, both mono and distearate, and the distearate containing less than about 7% of the mono stearate. When used, the suspending agent may be present in the compositions from about 0.1 wt. % to about 10 wt. %, and from about 0.3 wt. % to about 5.0 wt. %, by weight of the composition.

Thickeners

The compositions of the present invention may optionally include thickeners. Suitable thickeners may include, but are not limited to agar, agar-agar, guar gum, alginates, xanthan gum, gum arabic, gum karaya, locust bean flour, linseed gums, dextrans, cellulose derivatives (e.g., methylcellulose, hydroxyalkylcellulose, and carboxymethylcellulose), starch fractions and derivatives (e.g., amylose, amylopectin, and dextrins), clays (e.g., bentonite and montmorillonite), fully synthetic hydrocolloids (e.g., polyvinyl alcohol), polyacrylate polymers and co-polymers, (e.g., cross-linked polyacrylate copolymers such as Carbopol 940 available from Lubrizol), and mono- and di-valent salts (e.g., sodium chloride and potassium chloride). In particular embodiments the thickener comprises sodium chloride. When used, the thickener may be present in the compositions from about 0.1 wt. % to about 15 wt. %, and from about 1 wt. % to about 10 wt. %, by weight of the composition. In particular embodiments, the compositions of the invention may be substantially free of all thickeners or any particular type of thickener, such as a mono- or di-valent salt. In such embodiments, the thickeners may be present preferably at less than about 0.5 wt. %, more preferably at less than about 0.1 wt. %, and most preferably at less than about 0.01 wt. %.

Triglycerides

The compositions of the present invention may optionally include triglycerides. When used in the compositions of the invention, triglycerides may be present from about 0 wt. % to about 0.2 wt. %, from about 0.07 wt. % to about 0.15 wt. %, from about 0.08 wt. % to about 0.14 wt. %, and from about 0.09 wt. % to about 0.13 wt. %.

Vitamins and Amino Acids

The compositions of the present invention may optionally include vitamins and amino acids. Suitable vitamins may be water-soluble and/or water-insoluble. Examples of suitable vitamins for the compositions of the present invention include, but are not limited to, vitamins B1, B2, B6, B12, C, pantothenic acid, pantothenyl ethyl ether, panthenol, biotin and their derivatives, and vitamins A, D, E, and their derivatives. The compositions of the present invention may also contain water-soluble and water-insoluble amino acids such as asparagine, alanine, indole, glutamic acid and their salts, and tyrosine, tryptamine, lysine, histadine and their salts. When used in the compositions of the invention, vitamins and/or amino acids may be present from about 0 wt. % to about 2 wt. %, from about 0.05 wt. % to about 1 wt. %, and from about 0.09 wt. % to about 0.25 wt. %.

Additional Functional Ingredients

The components of the hair care product compositions can further be combined with various functional components suitable for application to human hair. Optionally, additional functional ingredients may be included in the compositions of the present invention. The functional ingredients provide desired properties and functionalities to the compositions. For the purpose of this application, the term “functional ingredient” includes a material that when dispersed or dissolved in a use and/or concentrate solution, such as an aqueous solution, provides a beneficial property in a particular use. Some particular examples of functional materials are discussed in more detail below, although the particular materials discussed are given by way of example only, and that a broad variety of other functional ingredients may be used. For example, many of the functional materials discussed below relate to materials used in hair care products, and may be particularly useful in shampoo, conditioner, styling gel, healing treatments, and/or hair spray. However, other embodiments may include functional ingredients for use in other applications.

Examples of functional ingredients, include, but are not limited to, abrasives, absorbents, aesthetic components such as perfumes and fragrances, pigments, colorings/colorants, essential oils, skin sensates, astringents (e.g., clove oil, menthol, camphor, eucalyptus oil, eugenol, menthyl lactate, witch hazel distillate), anti-acne agents, anti-caking agents, antifoaming agents, antimicrobial agents (e.g., iodopropyl butylcarbamate), antibacterial agents, antifungal agents, antioxidants, binders, biological additives, buffering agents, bulking agents, chemical additives, colorants, cosmetic astringents, cosmetic biocides, denaturants, drug astringents, external analgesics, opacifying agents, preservatives, propellants, reducing agents, sebum control agents, sequestrants, skin bleaching and lightening agents (e.g., hydroquinone, kojic acid, ascorbic acid, magnesium ascorbyl phosphate, ascorbyl glucoside, pyridoxine), enzymes, coenzymes, skin-conditioning agents (e.g., occlusive agents), skin soothing and/or healing agents and derivatives (e.g., ethyl panthenol, aloe vera, pantothenic acid and its derivatives, allantoin, bisabolol, and dipotassium glycyrrhizinate), skin treating agents (e.g., vitamin D compounds, mono-, di-, and tri-terpenoids, beta-ionol, cedrol), and combinations thereof.

### EMBODIMENTS

The compositions of the invention include from about preferably 9 mg/kg to about 13,750 mg/kg Osage Orange, more preferably from about 10 mg/kg to about 12,650 mg/kg Osage Orange, and most preferably from about 11 mg/kg to about 11,770 mg/kg Osage Orange. The compositions of the invention include preferably from about 35 wt. % to about 97.25 wt. % aqueous carrier, more preferably from about 40 wt. % to about 90 wt. % aqueous carrier, and most preferably from about 42.5 wt. % to about 85 wt. % aqueous carrier. The compositions of the invention include preferably from about 0.2 wt. % to about 3 wt. % antistatic agent, more preferably from about 0.3 wt. % to about 2.7 wt. % antistatic agent, and most preferably from about 0.35 wt. % to about 2.5 wt. % antistatic agent. The compositions of the invention include preferably from about 0.001 wt. % to about 20 wt. % conditioning agent, more preferably from about 0.01 wt. % to about 10 wt. % conditioning agent, and most preferably from about 0.1 wt. % to about 3 wt. % conditioning agent. The compositions of the invention include preferably from about 2 wt. % to about 50 wt. % surfactant, more preferably from about 2.5 wt. % to about 40 wt. % surfactant, and most preferably from about 3 wt. % to about 30 wt. % surfactant. The compositions of the invention include preferably from about 0.01 wt. % to about 2.5 wt. % total plant oil, or more preferably from about 0.02 wt. % to about 2.1 wt. % total plant oil. In addition, without being limited according to the invention, all ranges recited are inclusive of the numbers defining the range and include each integer within the defined range.

In certain embodiments of the invention, the compositions are substantially free of harsh chemical preservatives, including, chloroisothiazolinone, methylisothiazolinone, methylchloroisothiazolinone, sodium lauryl sulfate, tricolsan, and formaldehyde donors such as DMDM hydantoin when paired with iodopropynyl butylcarbamate or paraben. A still further object of the invention is to provide hair care products that are free of chemically synthesized anti-oxidants including, but not limited to, butylated hydroxyl anisole (BHA), butylated hydroxyl toluene (BHT), and tert-butyl hydroquinone (BHQ).

Applicant has found that some ratios of ingredients can be useful for ensuring easy dispensing from a bottle, uniform distribution in hair, and easy rinsing. So that the invention may be better understood and optimally practiced, some of those ratios are discussed below. In addition, without being limited according to the invention, all ranges for the ratios recited are inclusive of the numbers defining the range and include each integer within the defined range of ratios.

In an embodiment of the invention optimized for short, fine, or oily hair, the ratio of conditioning agent to antistatic agent is preferably less than about 1:1, more preferably from about 1:1 to about 1:3, even more preferably from about 1:1.5 to about 1:2.5, and most preferably at about 1:2. In some aspects of the invention the ratio of aqueous carrier to the combination of conditioning agent and antistatic agent is preferably from about 400:24 to about 750:24, more preferably from about 510:24 to about 720:24, and most preferably at about 680:24.

In another embodiment of the invention optimized for long, curly or dry hair, the ratio of conditioning agent to antistatic agent greater than about 1:1, preferably from about 1:1 to about 3:1, more preferably from about 1.25:1 to about 2:1, and even more preferable at about 1.5:1. In some aspects of the invention, the ratio of aqueous carrier to the combination of conditioning and antistatic agent is preferably from about 400:20 to about 750:20, and more preferably from about 500:20 to about 720:20, and most preferably at about 680:20. In another aspect of the invention the ratio of total plant oil to the combination of conditioning and antistatic agent is preferably from about 1:1 to about 4:5, more preferably from about 1:2 to about 3.5:5, and most preferably from about 1:3 to about 3:5. In an embodiment of the invention it is preferably about 1:3 for fine, limp, or oily hair and preferably about 3:5 for long or curly hair.

Viscosity

According to the present invention, the hair care products of the present invention, may have a viscosity of about 1,000 cps to about 300,000 cps, preferably from about 2,500 cps to about 250,000 cps, and most preferably from about 5,000 cps to about 200,000 cps. In an embodiment of the invention, a cleansing conditioner may have a viscosity preferably between about 50,000 cps and about 300,000 cps, more preferably between about 60,000 cps and about 250,000 cps, most preferably from about 80,000 to 200,000 cps. In an embodiment of the invention, a shampoo may have a viscosity preferably between about 1,000 cps and about 30,000 cps, more preferably between about 2,500 cps and about 25,000 cps, and most preferably between about 5,000 cps and about 20,000 cps.

### EXAMPLES

Embodiments of the present invention are further defined in the following non-limiting Examples. It should be understood that these Examples, while indicating certain embodiments of the invention, are given by way of illustration only. From the above discussion and these Examples, one skilled in the art can ascertain the essential characteristics of this invention, and without departing from the spirit and scope thereof, can make various changes and modifications of the embodiments of the invention to adapt it to various usages and conditions. Thus, various modifications of the embodiments of the invention, in addition to those shown and described herein, will be apparent to those skilled in the art from the foregoing description. Such modifications are also intended to fall within the scope of the appended claims.

The materials used in the following Examples are provided herein:

Carbopol 940 (2% aq.): a cross-linked polyacrylate copolymer available from Lubrizol.

Dow Corning® 193C Fluid: a dimethyl siloxane, available from Dow Corning.

Pomifera Extract: an Osage Orange extract in denatured ethanol, available from Osage Healthcare, Inc.

Pomifera Seed Oil: an Osage Orange seed oil, available from Osage Healthcare, Inc.

PVP/VA Luviskol® VA 73W: a polyvinylpyrrolidone/vinyl acetate copolymer, available from BASF.

Xiameter® MEM-8194 Emulsion: an amodimethicone blend, available from Dow Corning.

Additional materials commercially-available from multiple sources include: Aloe vera juice, avocado oil, behentrimonium chloride, cetearyl alcohol, cetyl alcohol, citric acid (10%), cocamide MEA, cocamidopropyl betaine (60%), colorant, cyclomethicone, dimethicone, EDTA tetrasodium, fragrance, glycerin, glycol stearate, grapeseed oil, jojoba oil, panthenol, PDS-500, PEG-40 hydrogenated castor oil, polysorbate 20, polysorbate 60, propane-1,2-diol, rice quat protein, shea butter, sodium chloride (20%), sodium cocoyl isethionate, sodium lauryl ether sulfate (60%), sodium lauryl sulfate (30%), stearamidopropyl dimethylamine, triethanolamine, triglycerides, and distilled water.

### Example 1

### Exemplary Cleansing Conditioners with Osage Orange Extract

An exemplary cleansing conditioner formulation is provided in Table A; it is particular beneficial for short, fine, or oily hair. This formula does not require pH adjustment to the acidic range typical of most conditioners or cleansing conditioners. As such, this embodiment can be substantially free of pH modifiers. The primary challenge that conditioners fail to address for this hair type is body and shine. Existing products trying to address an increase in shine typically leave this type of hair flat and/or heavy and without body. When body or deep moisturization is achieved with existing products the hair is left without shine, making the hair appear to have less fullness. This cleansing conditioner formula achieves the desired outcome for a conditioner or cleansing conditioner used on this type of hair i.e., full body and shine with deep moisturization.

TABLE A

| | Amount | | Amount per | |
|---|---|---|---|---|
| Component | (g) | Weight % | Total Mass | |
| Water | 510 | 73.30 | 733 | g/kg |
| PEG-40 Hydrogenated Castor Oil | 16 | 2.30 | 23.01 | g/kg |
| Cetyl Alcohol | 28 | 4.00 | 40.26 | g/kg |
| Cetearyl Alcohol | 22 | 3.20 | 31.64 | g/kg |
| Stearamidopropyl Dimethylamine | 8 | 1.20 | 11.50 | g/kg |
| Behentrimonium Chloride | 16 | 2.30 | 23.01 | g/kg |
| Glycerin | 36 | 5.20 | 51.77 | g/kg |
| Cocamidopropyl Betaine (60%) | 8 | 1.20 | 11.50 | g/kg |
| Grapeseed Oil | 8 | 1.20 | 11.50 | g/kg |
| Panthenol | 1 | 0.14 | 1.44 | g/kg |
| Polysorbate 60 | 4.8 | 0.69 | 6.90 | g/kg |
| Shea Butter | 4 | 0.58 | 5.75 | g/kg |
| Triglycerides | 0.8 | 0.12 | 1.15 | g/kg |
| Rice Quat Protein | 0.8 | 0.12 | 1.15 | g/kg |
| Dow Coming ® 193C Fluid | 1 | 0.14 | 1.44 | g/kg |
| Aloe Vera Juice | 30 | 4.30 | 43.14 | g/kg |
| Pomifera Extract | 1 | 0.14 | 1438 | mg/kg |

An exemplary cleansing conditioner formulation is provided in Table B; it is particular beneficial for long, curly, or dry hair. The primary challenge for this hair type is deep moisturization at the roots of the hair with concurrent detangling properties. This formulation addresses this need and leaves the root of the hair moisturized and detangled without loss of body or shine.

TABLE B

| | Amount | | Amount per | |
|---|---|---|---|---|
| Component | (g) | Weight % | Total Mass | |
| Water | 450 | 69.50 | 695 | g/kg |
| PEG-40 Hydrogenated Castor Oil | 16 | 2.50 | 24.71 | g/kg |
| Cetyl Alcohol | 28 | 4.30 | 43.25 | g/kg |
| Cetearyl Alcohol | 22 | 3.40 | 33.98 | g/kg |
| Stearamidopropyl Dimethylamine | 12 | 1.90 | 18.54 | g/kg |
| Behentrimonium Chloride | 8 | 1.20 | 12.36 | g/kg |
| Glycerin | 33 | 5.10 | 50.97 | g/kg |
| Cocamidopropyl Betaine (60%) | 8 | 1.20 | 12.36 | g/kg |
| Grapeseed Oil | 4 | 0.60 | 6.18 | g/kg |
| Avocado Oil | 4 | 0.60 | 6.18 | g/kg |
| Jojoba Oil | 4 | 0.60 | 6.18 | g/kg |
| Panthenol | 1 | 0.15 | 1.54 | g/kg |
| Polysorbate 60 | 4.8 | 0.74 | 7.41 | g/kg |
| Shea Butter | 4 | 0.62 | 6.18 | g/kg |
| Triglycerides | 0.8 | 0.12 | 1.24 | g/kg |
| Rice Quat Protein | 0.8 | 0.12 | 1.24 | g/kg |
| Dow Coming ® 193C Fluid | 1 | 0.15 | 1.54 | g/kg |
| Aloe Vera Juice | 45 | 7.00 | 69.51 | g/kg |
| Pomifera Extract | 1 | 0.15 | 1545 | mg/kg |

### Example 2

### Improved Texture and Rinsibility

Certain embodiments of the invention were found to have a texture best described as being somewhat waxy and at times hard to rinse out. Although these features did not prevent those formulations from providing shine, body, curl, fizz control, and moisturizing benefits, it is advantageous to also incorporate a smoothing texture and easier rinse properties into the cleansing conditioner.

It was determined that the addition of between about 0.1 wt. % and about 0.5 wt. % of the amphoteric surfactant, sodium cocoyl isethionate, dramatically changed the texture of the cleansing conditioner and made it easier to rinse out of the hair without loss of shine, body, curl, frizz control, and moisturizing benefits. It was also found that in certain embodiments more water could be added and the amount of waxy alcohols could be reduced without compromising performance of the cleansing conditioner. This provided the added benefit of cost reduction for such formulations.

Tables C and D provide exemplary formulations with improved texture and rinsibility and the added benefit of reduced manufacturing cost. In these embodiments, it is no longer necessary to reduce the pH to less than about 7.0. The reduction in cost can be partially attributed to the fact that the addition of a pH modifier is no longer necessary, nor is it necessary to add a sodium chloride to improve the viscosity at a lower pH. Thus, by eradicating the need of multiple chemicals, the cost for production is reduced while texture and rinsibility are improved. The exemplary formulation of Table C was found particularly beneficial for short, fine, or oily hair. The exemplary formulation of Table D was found particularly beneficial for long, curly, or dry hair.

TABLE C

| | | | Amount per | |
|---|---|---|---|---|
| | Amount | | Total Mass of | |
| Component | (g) | Weight % | Conditioner | |
| Water | 650 | 77.80 | 778 | g/kg |
| PEG-40 Hydrogenated Castor Oil | 16 | 1.90 | 19.15 | g/kg |
| Cetyl Alcohol | 26 | 3.10 | 31.12 | g/kg |
| Cetearyl Alcohol | 20 | 2.40 | 23.94 | g/kg |
| Sodium Cocoyl Isethionate | 4 | 0.50 | 4.79 | g/kg |
| Shea Butter | 4 | 0.50 | 4.79 | g/kg |
| Stearamidopropyl Dimethylamine | 8 | 1.00 | 9.58 | g/kg |
| Behentrimonium Chloride | 16 | 1.90 | 19.15 | g/kg |
| Glycerin | 36 | 4.30 | 43.09 | g/kg |
| Cocamidopropyl Betaine (60%) | 8 | 1.00 | 9.58 | g/kg |
| Pomifera Oil | 8 | 1.00 | 9.58 | g/kg |
| Aloe Vera Juice | 30 | 3.60 | 35.91 | g/kg |
| Panthenol | 1 | 0.12 | 1.20 | g/kg |
| Polysorbate 60 | 4.8 | 0.57 | 5.75 | g/kg |
| Triglycerides | 0.8 | 0.10 | 958 | mg/kg |
| Rice Quat Protein | 0.8 | 0.10 | 958 | mg/kg |
| Dow Coming ® 193C Fluid | 1 | 0.12 | 1.20 | g/kg |
| Pomifera Extract | 1 | 0.12 | 1197 | mg/kg |

TABLE D

| | | | Amount per | |
|---|---|---|---|---|
| | Amount | | Total Mass of | |
| Component | (g) | Weight % | Conditioner | |
| Water | 650 | 77.50 | 803 | g/kg |
| PEG-40 Hydrogenated Castor Oil | 16 | 1.90 | 19.77 | g/kg |
| Cetyl Alcohol | 28 | 3.30 | 34.59 | g/kg |
| Cetearyl Alcohol | 22 | 2.60 | 27.18 | g/kg |
| Sodium Cocoyl Isethionate | 4 | 0.50 | 4.94 | g/kg |
| Shea Butter | 4 | 0.50 | 4.94 | g/kg |
| Stearamidopropyl Dimethylamine | 12 | 1.40 | 14.83 | g/kg |
| Behentrimonium Chloride | 8 | 1.00 | 9.88 | g/kg |
| Glycerin | 36 | 4.30 | 44.48 | g/kg |
| Cocamidopropyl Betaine (60%) | 8 | 1.00 | 9.88 | g/kg |
| Pomifera Oil | 4 | 0.50 | 4.94 | g/kg |
| Avacado Oil | 4 | 0.50 | 4.94 | g/kg |
| Jojoba Oil | 4 | 0.50 | 4.94 | g/kg |
| Panthenol | 1 | 0.12 | 1.24 | g/kg |
| Polysorbate 60 | 4.8 | 0.57 | 5.93 | g/kg |
| Triglycerides | 0.8 | 0.10 | 988 | mg/kg |
| Rice Quat Protein | 0.8 | 0.10 | 988 | mg/kg |
| Dow Coming ® 193C Fluid | 1 | 0.12 | 1.24 | g/kg |
| Pomifera Extract | 1 | 0.12 | 1235 | mg/kg |

### Example 3

### Exemplary Cleansing Conditioners with Osage Orange Extract and Seed Oil

In some embodiments of the invention, it may be desirable to use both Osage Orange extract and Osage Orange seed oil. Exemplary formulations of a cleansing conditioner embodying the present invention and employing both Osage Orange extract and Osage Orange seed oil are provided in Tables E and F. The exemplary formulation in Table E is particularly beneficial for short, fine, or oily hair. The exemplary formulation in Table F is particularly beneficial for long, curly, or dry hair.

For both hair types each formula serves to open the hair cuticle such that it can be infused with humectants, Pomifera oil, and Osage Orange extract. The cuticle is then closed upon rinsing, leaving the hair full of body, moisture, shine, and very little frizz. The extract conveys the properties previously discussed while the oil has the additional advantage of providing moisturizing benefits, phytonutrients, and sun/light damage control due to its UV protective properties.

For fine, limp, oily hair the challenge is to provide body and shine without weighting the hair down—ultimately leaving it flat and without luster. This is achieved by the present invention. Thus, where traditional hair care products have left fine and limp hair greasy, lifeless, dull, or flat, the present invention has overcome the these problems. For example, where people with fine, limp hair would have had insufficient results when they used traditional hair care products, the hair care products of the present invention optimized for fine, limp, oily hair would achieve the desired results.

The long, curly formulation achieves the almost impossible—deep conditioning of dense, coarse hair at the scalp with concurrent moisturization at the ends of the hair. All while the hair is left full of body, moisture, shine, and very little frizz. For the long, curly formulation, excellent detangling properties were also achieved—very important to people with long, curly hair. Traditional hair care products have not been capable of achieving the results that the hair care products of the present invention optimized for long, curly achieve.

TABLE E

| | | | Amount per | |
|---|---|---|---|---|
| | Amount | | Total Mass of | |
| Component | (g) | Weight % | Conditioner | |
| Water | 650 | 77.80 | 778 | g/kg |
| PEG-40 Hydrogenated Castor Oil | 16 | 1.90 | 19.15 | g/kg |
| Cetyl Alcohol | 26 | 3.10 | 31.12 | g/kg |
| Cetearyl Alcohol | 20 | 2.40 | 23.94 | g/kg |
| Sodium Cocoyl Isethionate | 4 | 0.50 | 4.79 | g/kg |
| Shea Butter | 4 | 0.50 | 4.79 | g/kg |
| Stearamidopropyl Dimethylamine | 8 | 1.00 | 9.58 | g/kg |
| Behentrimonium Chloride | 16 | 1.90 | 19.15 | g/kg |
| Glycerin | 36 | 4.30 | 43.09 | g/kg |
| Cocamidopropyl Betaine (60%) | 8 | 1.00 | 9.58 | g/kg |
| Aloe Vera Juice | 30 | 3.60 | 35.91 | g/kg |
| Panthenol | 1 | 0.12 | 1.20 | g/kg |
| Polysorbate 60 | 4.8 | 0.57 | 5.75 | g/kg |
| Triglycerides | 0.8 | 0.10 | 958 | mg/kg |
| Rice Quat Protein | 0.8 | 0.10 | 958 | mg/kg |
| Dow Coming ® 193C Fluid | 1 | 0.12 | 1.20 | g/kg |
| Pomifera Seed Oil | 8 | 1.00 | 9576 | mg/kg |
| Pomifera Extract | 1 | 0.12 | 1197 | mg/kg |

TABLE F

| | | | Amount per | |
|---|---|---|---|---|
| | Amount | | Total Mass of | |
| Component | (g) | Weight % | Conditioner | |
| Water | 650 | 77.50 | 803 | g/kg |
| PEG-40 Hydrogenated Castor Oil | 16 | 1.90 | 19.77 | g/kg |
| Cetyl Alcohol | 28 | 3.30 | 34.59 | g/kg |
| Cetearyl Alcohol | 22 | 2.60 | 27.18 | g/kg |
| Sodium Cocoyl Isethionate | 4 | 0.50 | 4.94 | g/kg |
| Shea Butter | 4 | 0.50 | 4.94 | g/kg |
| Stearamidopropyl Dimethylamine | 12 | 1.40 | 14.83 | g/kg |
| Behentrimonium Chloride | 8 | 1.00 | 9.88 | g/kg |
| Glycerin | 36 | 4.30 | 44.48 | g/kg |
| Cocamidopropyl Betaine (60%) | 8 | 1.00 | 9.88 | g/kg |
| Grapeseed Oil | 4 | 0.50 | 4.94 | g/kg |
| Avacado Oil | 4 | 0.50 | 4.94 | g/kg |
| Panthenol | 1 | 0.12 | 1.24 | g/kg |
| Polysorbate 60 | 4.8 | 0.57 | 5.93 | g/kg |
| Triglycerides | 0.8 | 0.10 | 988 | mg/kg |
| Rice Quat Protein | 0.8 | 0.10 | 988 | mg/kg |
| Dow Coming ® 193C Fluid | 1 | 0.12 | 1.24 | g/kg |
| Pomifera Extract | 1 | 0.12 | 1235 | mg/kg |
| Pomifera Seed Oil | 4 | 0.50 | 4942 | mg/kg |

### Example 4

### Exemplary Shampoos with Osage Orange Extract and Seed Oil

Exemplary shampoo formulations were prepared using a combination of both Osage Orange extract and Osage Orange Seed Oil. Table G provides an exemplary shampoo formulation with excellent moisturizing, shine, and dry comb properties.

TABLE G

| | | | Amount per | |
|---|---|---|---|---|
| | Amount | | Total Mass of | |
| Component | (g) | Weight % | Shampoo | |
| Water | 506 | 57.20 | 572 | g/kg |
| Sodium Lauryl Sulfate (30%) | 110 | 12.40 | 124 | g/kg |
| Sodium Lauryl Ether Sulfate (60%) | 126 | 14.30 | 142 | g/kg |
| Cocamidopropyl Betaine (60%) | 16 | 1.80 | 18.10 | g/kg |
| Cocamide MEA | 16 | 1.80 | 18.10 | g/kg |
| EDTA Tetrasodium | 0.8 | 0.09 | 905 | mg/kg |
| Xiameter ® MEM-8194 Emulsion | 4 | 0.50 | 4.52 | g/kg |
| Glycol Stearate | 10 | 1.10 | 11.31 | g/kg |
| Behentriomium Chloride | 4 | 0.50 | 4.52 | g/kg |
| Sodium Chloride (20%) | 80 | 9.10 | 90.50 | g/kg |
| Citric Acid (10%) | 11 | 1.20 | 12.44 | g/kg |
| Pomifera Seed Oil | 0.1 | 0.01 | 113 | mg/kg |
| Pomifera Extract | 0.1 | 0.01 | 113 | mg/kg |

It was found that use of a larger amount of an amodimethicone blend provides a hydrating shampoo formulation that is pearlescent with a thick lather. Table H provides an exemplary shampoo formulation with excellent moisturizing, shine, and dry comb properties.

TABLE H

| | | | Amount per | |
|---|---|---|---|---|
| | Amount | | Total Mass of | |
| Component | (g) | Weight % | Shampoo | |
| Water | 506 | 56.00 | 560 | g/kg |
| Sodium Lauryl Sulfate (30%) | 110 | 12.20 | 122 | g/kg |
| Sodium Lauryl Ether Sulfate (60%) | 126 | 14.00 | 140 | g/kg |
| Cocamidopropyl Betaine (60%) | 16 | 1.80 | 17.72 | g/kg |
| Cocamide MEA | 16 | 1.80 | 17.72 | g/kg |
| EDTA Tetrasodium | 0.8 | 0.09 | 886 | mg/kg |
| Xiameter ® MEM-8194 Emulsion | 16 | 1.80 | 17.72 | g/kg |
| Glycol Stearate | 10 | 1.10 | 11.07 | g/kg |
| Behentriomium Chloride | 4 | 0.40 | 4430 | mg/kg |
| Sodium Chloride (20%) | 87 | 9.60 | 96.35 | g/kg |
| Citric Acid (10%) | 11 | 1.20 | 12.18 | g/kg |
| Pomifera Seed Oil | 0.1 | 0.01 | 111 | mg/kg |
| Pomifera Extract | 0.1 | 0.01 | 111 | mg/kg |

### Example 5

### Exemplary Formulations of Cleansing Conditioner and Methods of Preparation

An exemplary cleansing conditioner, particularly suitable for fine, limp, or oily hair was prepared with the ingredients in Table I. The cleansing conditioner was prepared by first measuring the ingredients of each Component. The ingredients in Component A were charged at room temperature with stirring and then heated to 85° C. and maintained at 85° C. for 5 minutes. The 85° C. Component A was poured into the container holding Component B at 25° C. This mixture was stirred and allowed to cool to 40° C. without heat control. When the temperature of the mixture reached 40° C., Component C was added to the mixture of Components A and B. The mixture was then stirred until it reached room temperature.

An exemplary cleansing conditioner, particularly suitable for curly hair was prepared with the ingredients in Table J. The cleansing conditioner was prepared by first measuring the ingredients of each Component. The ingredients in Component A were charged at room temperature with stirring and then heated to 85° C. and maintained at 85° C. for 5 minutes. The 85° C. Component A was poured into the container holding Component B at 25° C. This mixture was stirred and allowed to cool to 40° C. without heat control. When the temperature of the mixture reached 40° C., Component C was added to the mixture of Components A and B. The mixture was then stirred until it reached room temperature.

TABLE I

| | | Amount per Total | |
|---|---|---|---|
| Ingredients | Amount (g) | Mass of Conditioner | |
| Component A | | | |
| Water | 1200 | 719.977 | g/kg |
| PEG-40 Hydrogenated Castor Oil | 32 | 19.199 | g/kg |
| Cetyl Alcohol | 52 | 31.199 | g/kg |
| Cetearyl Alcohol | 40 | 23.999 | g/kg |
| Sodium Cocoyl Isethionate | 8 | 4.800 | g/kg |
| Shea Butter | 8 | 4.800 | g/kg |
| Stearamidopropyl Dimethylamine | 16 | 9.600 | g/kg |
| Behentrimonium Chloride | 32 | 19.199 | g/kg |
| Component B | | | |
| Glycerin | 72 | 43.199 | g/kg |
| Cocamidopropyl Betaine | 16 | 9.600 | g/kg |
| Pomifera Oil | 16 | 9.600 | g/kg |
| Grapeseed Oil | 0.01 | 6.000 | mg/kg |
| Water | 160 | 95.997 | g/kg |
| Panthenol | 0.1 | 59.998 | mg/kg |
| Polysorbate 60 | 9.6 | 5.760 | g/kg |
| Triglycerides | 1 | 599.980 | mg/kg |
| Rice Quat Protein | 1 | 599.980 | mg/kg |
| Dow Coming ® 193C Fluid | 3 | 1.800 | g/kg |
| Component C | | | |
| Pomifera Extract | 1.0 | 599 | mg/kg |
| Total Mass of Cleansing Conditioner | 1667.71 | | |

TABLE J

| | | Amount per Total | |
|---|---|---|---|
| Ingredient | Amount (g) | Mass of Conditioner | |
| Component A | | | |
| Water | 1200 | 716.542 | g/kg |
| PEG-40 Hydrogenated Castor Oil | 32 | 19.108 | g/kg |
| Cetyl Alcohol | 56 | 33.439 | g/kg |
| Cetearyl Alcohol | 44 | 26.273 | g/kg |
| Sodium Cocoyl Isethionate | 8 | 4.777 | g/kg |
| Shea Butter | 8 | 4.777 | g/kg |
| Stearamidopropyl Dimethylamine | 24 | 14.331 | g/kg |
| Behentrimonium Chloride | 15 | 8.957 | g/kg |
| Component B | | | |
| Glycerin | 72 | 42.993 | g/kg |
| Cocamidopropyl Betaine | 16 | 9.554 | g/kg |
| Pomifera Oil | 8 | 4.777 | g/kg |
| Avocado Oil | 8 | 4.777 | g/kg |
| Grapeseed Oil | 0.01 | 5.971 | mg/kg |
| Jojoba Oil | 8 | 4.777 | g/kg |
| Water | 160 | 95.539 | g/kg |
| Panthenol | 0.1 | 59.712 | mg/kg |
| Polysorbate 60 | 9.6 | 5.732 | g/kg |
| Triglycerides | 1 | 597.118 | mg/kg |
| Rice Quat Protein | 1 | 597.118 | mg/kg |
| Dow Coming ® 193C Fluid | 3 | 1.791 | g/kg |
| Component C | | | |
| Pomifera Extract | 1 | 597.118 | mg/kg |
| Total Mass of Cleansing Conditioner | 1674.71 | | |

### Example 6

### Exemplary Formulation of Styling Gel and Method of Preparation

An exemplary styling gel was prepared with the ingredients in Table K. The styling gel was prepared by first measuring the ingredients of each Component. Component A was stirred at 25° C. until uniform. Then Component B was slowly added to Component A with agitation. The agitation was adjusted to achieve a uniform viscous gel. Sequentially, the remaining components were added to the mixture. Before the addition of a subsequent component, the mixture was mixed until uniformity of the mixture was achieved.

TABLE K

| | | Amount per Total | |
|---|---|---|---|
| Ingredient | Amount (g) | Mass of Conditioner | |
| Component A | | | |
| Water | 45.3 | 453.0 | g/kg |
| Carbopol 940 | 25.0 | 250.0 | g/kg |
| Component B | | | |
| Water | 0.5 | 5.0 | g/kg |
| Triethanolamine | 0.6 | 6.0 | g/kg |
| Pomifera Extract | 1.0 | 10,000.0 | mg/kg |
| Pomifera Oil | 0.1 | 1000.0 | mg/kg |
| Component C | | | |
| Glycerin | 15.0 | 150.0 | g/kg |
| Component D | | | |
| PVP/VA Luviskol ® VA 73W | 10.0 | 100.0 | g/kg |
| Component E | | | |
| Propane-1,2-diol | 1.0 | 10.0 | g/kg |
| Component F | | | |
| Polysorbate 20 | 1.0 | 10.0 | g/kg |
| Fragrance | 0.5 | 5.0 | g/kg |
| Total Mass of Styling Gel | 100.00 | | |

### Example 7

### Exemplary Formulation of Healing Treatment and Method of Preparation

An exemplary healing treatment was prepared with the ingredients in Table L. The healing treatment was prepared by first measuring the ingredients of each Component. The components were sequentially mixed at 25° C., making sure uniformity of the mixture was achieved before adding the next component.

TABLE L

| | | Amount per Total | |
|---|---|---|---|
| Ingredient | Amount (g) | Mass of Conditioner | |
| Component A | | | |
| Dimethicone | 50 | 500 | g/kg |
| Component B | | | |
| Cyclomethicone | 49.4 | 494.0 | g/kg |
| Component C | | | |
| Pomifera Oil | 0.2 | 2000.0 | mg/kg |
| Component D | | | |
| Fragrance | 0.2 | 2.0 | g/kg |
| Component E | | | |
| Colorant | 0.2 | 2.0 | g/kg |
| Total Mass of Healing Treatment | 100.00 | | |

The inventions being thus described, it will be understood that the same may be varied in many ways. Such variations are not to be regarded as a departure from the spirit and scope of the inventions and all such modifications are intended to be included within the scope of the following claims.

1. A hair care product comprising:
plant oil comprising Osage Orange, wherein said Osage Orange is in the form of a botanical extract, seed oil, or a combination thereof;
butter between 0.05 wt. % and about 5 wt. % of the hair care product;
antistatic agent comprising a cationic quaternary ammonium;
conditioning agent comprising a fatty amine;
surfactant; and
an aqueous carrier;
wherein the ratio of conditioning agent to antistatic agent is between about 1.25:1 and about 2:1 by weight percent; and wherein the ratio of water to the combination of conditioning agent and antistatic agent is from about 500:20 to about 720:20 by weight percent.

2. The hair care product of claim 1, wherein the Osage Orange is present in the composition between about 9 mg/kg to about 13,750 mg/kg of the total composition.

3. The hair care product of claim 1, wherein the ratio of conditioning agent to antistatic agent is about 1.5:1 by weight percent.

4. The hair care product of claim 1, wherein the aqueous carrier comprises water and an organic solvent.

5. The hair care product of claim 4, wherein the ratio of water to the combination of conditioning agent and antistatic agent is about 680:20 by weight percent.

6. The hair care product of claim 4, wherein the ratio of total plant oil to the combination of conditioning agent and antistatic agent is about 1:2 to about 3.5:5 by weight percent.

7. The hair care product of claim 6, wherein the ratio of total plant oil to the combination of conditioning agent and antistatic agent is about 3:5 by weight percent.

8. The hair care product of claim 1 further comprising at least one of the following: an additional plant-derived ingredient, a chelating agent, an emulsifier, a fatty protein, a silicone, a thickener, triglycerides, or vitamins.

9. The hair care product of claim 1, wherein the cationic quaternary ammonium compound comprises behentrimonium chloride, behentrimonium methosulfate, or a combination thereof; and wherein the fatty amine comprises stearamidopropyl dimethylamine.

10. A hair care product for short, fine, or oily hair comprising:
plant oil comprising Osage Orange, wherein said Osage Orange is in the form of a botanical extract, seed oil, or a combination thereof, wherein the Osage Orange is present between about 9 mg/kg and about 15 mg/kg of the hair care product;
butter comprising at least one of the following cocoa butter, cupuacu butter, goa butter, mango butter, shea butter, or mixtures thereof, wherein the butter is between about 0.05 wt. % and about 4 wt. %;
antistatic agent comprising a cationic quaternary ammonium, wherein the antistatic agent is present between about 0.2 wt. % and about 3 wt. %;
conditioning agent comprising a fatty amine, wherein the conditioning agent is present between about 0.001 wt. % to about 20 wt. %;
surfactant present between about 2 wt. % and about 50 wt. % of the hair care product, wherein the surfactant comprises a zwitterionic surfactant; and
an aqueous carrier present between about 40 wt. % and about 95 wt. % of the hair care product;
wherein the ratio of conditioning agent to antistatic agent is from about 1:1.5 to about 1:2.5 by weight percent, and wherein the ratio of aqueous carrier to the combination of conditioning agent and antistatic agent is between about 510:24 and about 720:24 by weight percent.

11. The hair care product of claim 10, wherein the Osage Orange is a combination of botanical extract and seed oil.

12. The hair care product of claim 10, wherein the ratio of conditioning agent to antistatic agent is about 1:2 by weight percent.

13. The hair care product of claim 10, wherein the ratio of aqueous carrier to the combination of conditioning agent and antistatic agent is about 680:24 by weight percent.

14. The hair care product of claim 10, wherein the composition is substantially free of harsh chemicals and chemically synthesized antioxidants.

15. A hair care product long, curly, or dry hair comprising:
plant oil comprising Osage Orange, wherein the Osage Orange is in the form of a botanical extract, seed oil, or a combination thereof, wherein the Osage Orange is present between about 445 mg/kg and about 75 mg/kg of the hair care product;
butter comprising at least one of the following cocoa butter, cupuacu butter, goa butter, mango butter, shea butter, or mixtures thereof, wherein the butter is between about 0.05 wt. % and about 4 wt. %;
antistatic agent comprising a cationic quaternary ammonium, wherein the antistatic agent is present between about 0.2 wt % and about 3 wt. %;
conditioning agent comprising a fatty amine, wherein the conditioning agent is present between about 0.001 wt. % to about 20 wt. %;
surfactant present between about 2 wt. % and about 50 wt. % of the hair care product, wherein the surfactant comprises a zwitterionic surfactant; and
an aqueous carrier present between about 40 wt. % and about 95 wt. % of the hair care product;
wherein the ratio of conditioning agent to antistatic agent is about 1:2 or about 1.5:1 by weight percent, and wherein the ratio of aqueous carrier to the combination of conditioning agent and antistatic agent is about 680:24 or about 680:20 by weight percent.

16. The hair care product of claim 15, wherein the ratio of total plant oil to the combination of conditioning agent and antistatic agent is between about 1:2 and about 3.5:5 by weight percent.

17. The hair care product of claim 15, wherein the Osage Orange is present between about 500 mg/kg and about 690 mg/kg of the hair care product.

18. The hair care product of claim 15, wherein the Osage Orange is present between about 550 mg/kg and about 640 mg/kg of the hair care product, and wherein the ratio of total plant oil to the combination of conditioning agent and antistatic agent is about 1:3 or about 3:5 by weight percent.

19. The hair care product of claim 15, wherein the cationic quaternary ammonium compound comprises behentrimonium chloride, behentrimonium methosulfate, or a combination thereof; and wherein the fatty amine comprises stearamidopropyl dimethylamine.

20. The hair care product of claim 15, wherein the composition is substantially free of harsh chemicals and chemically synthesized antioxidants.

21. A styling gel comprising:
Osage Orange in the form of a botanical extract, seed oil, or a combination thereof, wherein the Osage Orange is present between about 8,250 mg/kg and about 13,750 mg/kg of the styling gel;
a fixative present between about 0 wt. % and about 20 wt. %;
an aqueous carrier;
a hydrotrope present between about 0.01 wt. % and about 10 wt. %;
an emulsifier present between about 0 wt. % and about 5 wt. %; and
a thickener present between about 0.1 wt. % and about 15 wt. %.

22. The styling gel of claim 21, wherein the composition is substantially free of a pH modifier and monovalent and divalent salts.

23. The styling gel of claim 21 further comprising fragrance.

24. A healing treatment for hair comprising:
Osage Orange in the form of a botanical extract, seed oil, or a combination thereof, wherein the Osage Orange is present between about 1,500 mg/kg and about 2,500 mg/kg of the healing treatment;
a silicone compound from about 74.5 wt. % to about 99.9 wt. %;
at least one additional functional ingredient selected from the group consisting essentially of perfumes, fragrances, pigments, colorants, and combinations thereof.

25. The healing treatment of claim 24, wherein the silicone compound is selected from the group consisting essentially of cylcomethicones, dimethicones, amodimethicones, amodimethicone blends, and combinations thereof.
